(19) United States
(12) Patent Application Publication
(10) Pub. No.: US 2008/0090771 A1
(43) Pub. Date: Apr. 17, 2008

(54) ABUSE-RESISTANT HYDROCODONE COMPOUNDS, COMPOSITIONS AND METHODS OF USING THE SAME

(75) Inventor: James Scott Moncrief, Christiansburg, VA (US)

Correspondence Address: DARBY & DARBY P.C., P.O. BOX 770, Church Street Station, New York, NY, 10008-0770, US

(73) Assignee: SHIRE LLC, Florence, KY (US)

(21) Appl. No.: 11/868,636

(22) Filed: Oct. 8, 2007

Related U.S. Application Data
(60) Provisional application No. 60/849,776, filed on Oct. 6, 2006.

(51) Int. Cl. A61K 31/4355; A61K 38/05; A61K 38/06; A61K 38/08; A61P 25/00; C07D 471/04; C07K 5/062; C07K 5/083; C07K 7/06

(52) U.S. Cl. 514/17; 514/18; 514/19; 514/282; 530/300; 530/330; 530/331; 546/45

(57) ABSTRACT

The invention relates to compounds, compositions and methods comprised of a chemical moiety attached to hydrocodone. The invention provides embodiments that provide a decrease in the potential of hydrocodone to cause overdose or to be abused while still delivering therapeutic activity similar to that of the parent hydrocodone. The invention also provides methods of delivering hydrocodone as conjugates that release the hydrocodone following oral administration while being resistant to abuse by other routes such as intravenous injection (“shooting”) and intranasal administration (“snorting”). Further, hydrocodone compositions of the invention are resistant to oral abuse as well, since release of the hydrocodone at suprapharmacological doses reaches saturation.

This application claims priority under 35 U.S.C. §119(e) to U.S. provisional application Ser. No. 60/849,776 filed Oct. 6, 2006.

### FIELD OF INVENTION

The invention relates to pharmaceutical compounds, compositions and methods of using the same comprising a chemical moiety attached to hydrocodone. These inventions provide a variety of beneficial effects. Some inventions result in a substantial decreases the potential of hydrocodone to cause overdose or to be abused. For instance, some inventions provide therapeutic activity similar to that of the parent hydrocodone when delivered at typical dosage ranges, however when delivered at higher doses the potential for overdose or abuse is reduced due to the limited bioavailability of hydrocodone as compared to hydrocodone delivered in an non-conjugated form. Alternatively or in addition, the prodrug may be designed to provide fast or slow release depending of its use for chronic pain versus acute pain. Additionally, some of the inventions may reduce the side effects associated with taking hydrocodone.

Opioids are highly effective as analgesics and are commonly prescribed for the treatment of acute and chronic pain. They are also commonly used as antitussives. The opioids, however, also produce euphoria and are highly addictive. As a result they are often abused with far reaching social and health related consequences. The present invention decreases the potential for abuse of opioids, particularly hydrocodone, by covalent modification. The invention provides methods of delivering hydrocodone as conjugates that release the hydrocodone following oral administration while being resistant to abuse by circuitous routes such as intravenous (“shooting”) injection and intranasal administration (“snorting”). Further, hydrocodone compositions of the invention are resistant to oral abuse as well, since release of the hydrocodone at suprapharmacological doses reaches saturation. The invention also decreases the chances of dose escalation that often leads to accidental addiction.

### BACKGROUND

Despite their addictive properties and the potential for abuse, morphine-like drugs, particularly, codeine, hydrocodone, and oxycodone have been routinely prescribed as treatment for severe acute and chronic pain in recent decades. This is, in part, because there are no alternatives to relieve severe pain that is resistant to other less potent analgesics such as non-steroidal anti-inflammatory drugs (NSAIDS). In this regard, others have attempted to decrease the abuse potential through formulations and the inclusion of morphine antagonists such as naltrexone. These approaches, unfortunately, can be circumvented and have not solved the problem.

In recent years the misuse of opioid painkillers has nearly quadrupled. An estimated 2.4 million people in the U.S. began misusing prescription pain killers in 2001 as compared to 628,000 in 1990 according to the federal government's Survey on Drug Use and Health. An estimated 4.4 million patients take more pain medication than their prescribed amount. The rate of full blown addiction is 0.3 percent, however, any patient that does not follow their prescription is considered at risk. Pain medications prescribed for acute pain typically contain about 5 to 10 mg of hydrocodone, oxycodone, or codeine.

Hydrocodone is an opioid analgesic and antitussive and occurs as fine, white crystals or as crystalline powder. Hydrocodone is a semisynthetic narcotic analgesic prepared from codeine with multiple actions qualitatively similar to those of codeine. It is mainly used as an antitussive in cough syrups and tablets in sub-analgesic doses (2.5-5 mg). Additionally, it is used for the relief of moderate to moderately severe pain. Patients taking opioid analgesics such as hydrocodone for pain relief can become accidentally addicted. As tolerance to the opioids develops more drug is needed to stop the pain and generate the sense of well being initially achieved with the prescribed dose. This leads to dose escalation, which if left unchecked can lead rapidly to addiction. In some cases patients have become full blown addicts in as little as thirty days.

As a result of their addictive properties and potential for abuse, opioids are scheduled controlled substances and are available only by prescription. It has been suggested that this precipitates under-utilization of opioids for pain relief. Although it is well known that opioids are the most effective treatment for severe pain, their abuse liability and the potential for fatal overdose provide a legitimate concern for any physician considering their use in pain management.

Consequently, improved methods are needed to make pharmaceutically effective hydrocodone compounds, compositions and methods of using the same with reduced potential for overdose and/or resistance to manipulation while still providing necessary analgesia for various types of pain. Preferably, absorption of the composition into the brain is prevented or substantially diminished and/or delayed when delivered by routes other than oral administration.

### BRIEF DESCRIPTION OF DRAWINGS

FIG. 1 depicts the numbering scheme for hydrocodone.

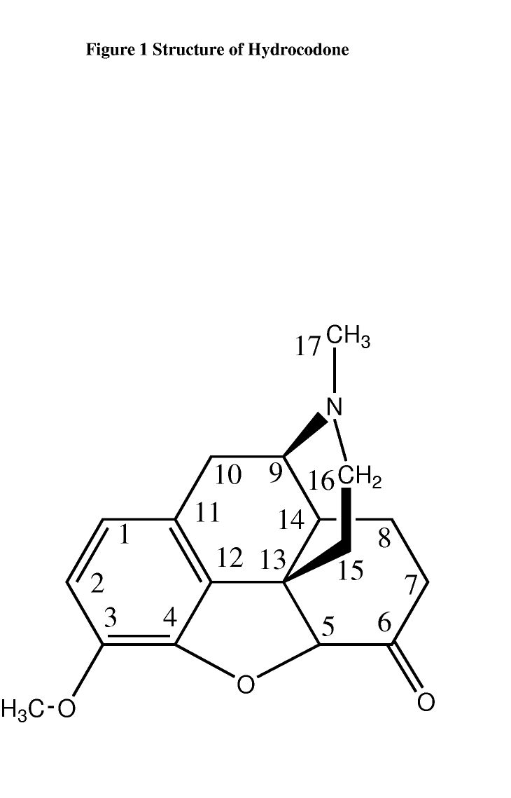

FIG. 2 depicts hydrocodone conjugated at the 6 position.

### DETAILED DESCRIPTION OF THE INVENTION

The invention relates to changing the pharmacokinetic and pharmacological properties of hydrocodone through covalent modification. Covalent attachment of a chemical moiety to hydrocodone may change one or more of the following: the rate of absorption, the extent of absorption, the metabolism, the distribution, and the elimination (ADME pharmacokinetic properties) of hydrocodone. As such, the alteration of one or more of these characteristics may be designed to provide fast or slow release depending of its use for chronic pain versus acute pain. Additionally, alteration of one or more of these characteristics may reduce the side effects associated with taking hydrocodone

One aspect of the invention includes hydrocodone conjugates that when administered at a normal therapeutic dose the bioavailability (area under the time-versus-concentration curve; AUC) of hydrocodone provides a pharmaceutically effective amount of hydrocodone. As the dose is increased, however, the bioavailability of the covalently modified hydrocodone relative to the parent hydrocodone begins to decline, particularly for oral dosage forms. At suprapharmacological doses the bioavailability of the hydrocodone conjugate is substantially decreased as compared to the parent hydrocodone. The relative decrease in bioavailability at higher doses decreases or reduces the euphoria obtained when doses of the hydrocodone conjugate are taken above those of the intended prescription. This in turn diminishes the abuse potential, whether unintended or intentionally sought.

The invention provides hydrocodone prodrugs comprising hydrocodone covalently bound to a chemical moiety. The hydrocodone prodrugs can also be characterized as conjugates in that they possess a covalent attachment. They may also be characterized as conditionally bioreversible derivatives (“CBDs”).

In one embodiment, the hydrocodone prodrug (a compound of one of the formulas described herein) may exhibit one or more of the following advantages over free hydrocodone. The hydrocodone prodrug may prevent overdose by exhibiting a reduced pharmacological activity when administered at higher than therapeutic doses, e.g., higher than the prescribed dose. Yet when the hydrocodone prodrug is administered at therapeutic doses, the hydrocodone prodrug may retain similar pharmacological activity to that achieved by administering unbound hydrocodone. Also, the hydrocodone prodrug may prevent abuse by exhibiting stability under conditions likely to be employed by illicit chemists attempting to release the hydrocodone. The hydrocodone prodrug may prevent abuse by exhibiting reduced bioavailability when it is administered via parenteral routes, particularly the intravenous (“shooting”), intranasal (“snorting”), and/or inhalation (“smoking”) routes that are often employed in illicit use. Thus, the hydrocodone prodrug may reduce the euphoric effect associated with hydrocodone abuse. Thus, the hydrocodone prodrug may prevent and/or reduce the potential of abuse and/or overdose when the hydrocodone prodrug is used in a manner inconsistent with the manufacturer's instructions, e.g., consuming the hydrocodone prodrug at a higher than therapeutic dose or via a non-oral route of administration.

Preferably, the hydrocodone prodrug provides a serum release curve that does not increase above the toxicity level of hydrocodone when administered at higher than therapeutic doses. The hydrocodone prodrug may exhibit a reduced rate of hydrocodone absorption and/or an increased rate of clearance compared to the free hydrocodone. The hydrocodone prodrug may also exhibit a steady-state serum release curve. Preferably, the hydrocodone prodrug provides bioavailability but prevents Cₘₐₓ spiking or increased blood serum concentrations.

Hydrocodone may be bound to one or more chemical moieties, denominated X and Z. A chemical moiety can be any moiety that decreases the pharmacological activity of hydrocodone while bound to the chemical moiety as compared to unbound (free) hydrocodone. The attached chemical moiety can be either naturally occurring or synthetic. In one embodiment, the invention provides an hydrocodone prodrug of Formula IA or IB:
H-XnₙZₘ (IA)
H-Zₘ-Xₙ (IB)
wherein H is an hydrocodone;
each X is independently a chemical moiety;
each Z is independently a chemical moiety that acts as an adjuvant and is different from at least one X;
n is an increment from 1 to 50, preferably 1 to 10; and
m is an increment from 0 to 50, preferably 0.
When m is 0, the hydrocodone prodrug is a compound of Formula (II):
H—Xₙ (II)
wherein each X is independently a chemical moiety.

Formula (II) can also be written to designate the chemical moiety that is physically attached to the hydrocodone:
H—X₁-(X)ₙ₋₁ (III)
wherein H is hydrocodone; X₁ is a chemical moiety, preferably a single amino acid; each X is independently a chemical moiety that is the same as or different from X₁; and n is an increment from 1 to 50.

H is hydrocodone and has the following structure where substitution occurs at the 6 position of hydrocodone wherein A represents the attachment site for X.

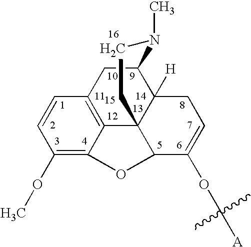

In an alternative embodiment, the 3 position and/or the N position of hydrocodone may be substituted with a chemical moiety with or without the presence of a linker. See U.S. Pat. No. 5,610,283 for methods of substituting opioids at these positions. Chemical moieties include, but are not limited to any of the carrier peptides listed below in Table 1.

Compounds, compositions and methods of the invention provide reduced potential for overdose, reduced potential for abuse or addiction and/or improve the characteristics of hydrocodone with regard to high toxicities or suboptimal release profiles. Without wishing to be limited to the below theory, we believe that in some instances overdose protection results from a natural gating mechanism at the site of hydrolysis that limits the release of hydrocodone from the prodrug at greater than therapeutically prescribed amounts. Therefore, abuse resistance is provided by limiting the “rush” or “high” available from the hydrocodone released by the prodrug and limiting the effectiveness of alternative routes of administration for certain chemical moieties.

The invention utilizes covalent modification of hydrocodone to alter its ADME for certain delivery routes, e.g. routes other than oral, to decrease its potential for causing overdose or being abused. The hydrocodone is covalently modified in a manner that decreases its pharmacological activity, as compared to the unmodified hydrocodone, at doses above those considered therapeutic, e.g., at doses inconsistent with the manufacturer's instructions. When given at lower doses, such as those intended for therapy, covalently modified hydrocodone retains effective pharmacological activity. The covalent modification of hydrocodone may comprise the attachment of any chemical moiety through conventional chemistry. Preferably the chemical moiety is a carrier peptide.

Further, at times the invention is described as being hydrocodone attached to an amino acid, a dipeptide, a tripeptide, tetrapeptide, pentapeptide, or hexapeptide to illustrate specific embodiments for the hydrocodone conjugate. Preferred lengths of the conjugates and other preferred embodiments are described herein. Preferred carriers are listed in Tables 1 and 2.

Persons that abuse prescription drugs commonly seek to increase their euphoria by snorting or injecting the drugs. These routes of administration increase the rate and extent of drug absorption and provide a faster, nearly instantaneous, effect. This increases the amount of drug that reaches the central nervous system where it has its effect. In a particular embodiment of the invention the bioavailability of the covalently modified hydrocodone is substantially decreased when taken by the intranasal and intravenous routes as compared to the parent hydrocodone. Thus the illicit practice of snorting and shooting the drug loses its advantage, i.e., the central nervous system effects are diminished.

In another embodiment of the invention, the solubility and dissolution rate of the composition is substantially changed under physiological conditions encountered in the intestine, at mucosal surfaces, or in the bloodstream. In another embodiment the solubility and dissolution rate substantially decrease the bioavailability of the hydrocodone prodrug, particularly at doses above those intended for therapy. In another embodiment the decrease in bioavailability occurs upon oral administration. In another embodiment the decrease in bioavailability occurs upon intranasal administration. In another embodiment the decrease in bioavailability occurs upon intravenous administration.

Another particular embodiment of the invention provides that when the covalently modified hydrocodone is provided in oral dosage form (e.g., a tablet, capsule, caplet, liquid dispersion, etc.) it has increased resistance to manipulation. For, instance, crushing of a tablet or disruption of a capsule does not substantially increase the rate and amount of hydrocodone absorbed when compositions of the invention are ingested.

Another embodiment of the invention provides compositions and methods of providing analgesia comprising administering to a patient compounds or compositions of the invention. Another embodiment provides a composition or method for treating pain in a patient i.e., acute and chronic pain—it should be noted that different conjugates maybe be utilized to treat acute versus chronic pain.

Hydrocodone may be attached to the carrier peptide through the C-terminus, N-terminus, or side chain of the carrier peptide. Preferably, hydrocodone is attached to the C-terminus of the carrier peptide. It is preferred that aside from attachment of the carrier peptide to the hydrocodone neither is further substituted or protected. In one embodiment, the chemical moiety has one or more free carboxy and/or amine terminus and/or side chain group other than the point of attachment to the hydrocodone. The chemical moiety can be in such a free state, or an ester or salt thereof.

Another embodiment of the invention is a composition or method for safely delivering hydrocodone comprising providing a therapeutically effective amount of said hydrocodone which has been covalently bound to a chemical moiety wherein said chemical moiety reduces the rate of absorption of the hydrocodone as compared to delivering the unbound hydrocodone.

Another embodiment of the invention is a composition or method for reducing drug toxicity comprising providing a patient with hydrocodone which has been covalently bound to a chemical moiety wherein said chemical moiety increases the rate of clearance of hydrocodone when given at doses exceeding those within the therapeutic range of said hydrocodone.

Another embodiment provides a composition or method of reducing drug toxicity comprising providing a patient with hydrocodone which has been covalently bound to a chemical moiety wherein the chemical moiety provides a serum release curve which does not increase above the toxicity level of hydrocodone when given at doses exceeding those within the therapeutic range for unbound hydrocodone.

Another embodiment provides a composition that reduces or eliminates the toxic range of the Lethal Dose, 50% (LD₅₀) comprising providing a composition containing hydrocodone, which has been covalently bound to a chemical moiety.

Another embodiment of the invention is a composition or method for a sustained-release hydrocodone composition comprising providing hydrocodone which has been covalently bound to a chemical moiety, wherein said chemical moiety provides release of hydrocodone at a rate where the level of hydrocodone is within the therapeutic range but below toxic levels over an extended periods of time, e.g., 8-24 hours or greater.

Another embodiment of the invention is a composition or method for reducing bioavailability or preventing a toxic release profile of hydrocodone comprising hydrocodone covalently bound to a chemical moiety wherein said bound hydrocodone maintains a steady-state serum release curve which provides a therapeutically effective bioavailability but prevents spiking or increase blood serum concentrations compared to unbound hydrocodone when given at doses exceeding those within the therapeutic range of said hydrocodone.

Another embodiment of the invention is a composition or method for preventing a Cₘₐₓ spike for hydrocodone while still providing a therapeutically effective bioavailability curve comprising hydrocodone which has been covalently bound to a chemical moiety.

In another embodiment the compositions have substantially lower toxicity compared to unbound hydrocodone. In another embodiment the compositions reduce or eliminate the possibility of overdose by oral administration. In another embodiment the compositions reduce or eliminate the possibility of overdose by intranasal administration. In another embodiment the compositions reduce or eliminate the possibility of overdose by injection.

The invention further provides compositions or methods for altering hydrocodone in a manner that decreases their potential for abuse. Compositions and methods of the invention provide various ways to regulate pharmaceutical dosage through covalent attachment of hydrocodone to different chemical moieties. One embodiment provides a method of preventing overdose comprising administering to an individual hydrocodone which has been covalently bound to a chemical moiety.

Another embodiment of the invention is a method for reducing or preventing abuse or euphoric effect of a pharmaceutical composition, comprising providing, administering, or prescribing said composition to a human in need thereof, wherein said composition comprises a chemical moiety covalently attached to hydrocodone such that the pharmacological activity of hydrocodone is substantially decreased when the composition is used in a manner inconsistent with the manufacturer's instructions or in a manner that substantially increases the potential of overdose from hydrocodone.

Another embodiment of the invention is a method for reducing or preventing abuse or euphoric effect of a pharmaceutical composition, comprising consuming said composition, wherein said composition comprises a chemical moiety covalently attached to hydrocodone such that the pharmacological activity of hydrocodone is substantially decreased when the composition is used in a manner inconsistent with the manufacturer's instructions or in a manner that substantially decreases the potential of overdose from hydrocodone.

Another embodiment of the invention is any of the preceding methods wherein said pharmaceutical composition is adapted for oral administration, and wherein said hydrocodone is resistant to release from said chemical moiety when the composition is administered parenterally, such as intranasally or intravenously. Preferably, said hydrocodone may be released from said chemical moiety in the presence of acid and/or enzymes present in the stomach, intestinal tract, or blood serum.

Another embodiment of the invention is any of the herein described methods wherein said composition yields a therapeutic effect without substantial euphoria. Preferably, said hydrocodone provides a therapeutically bioequivalent AUC when compared to hydrocodone alone but does not provide a Cₘₐₓ which results in euphoria.

Another embodiment of the invention is a method for reducing or preventing abuse of a pharmaceutical composition, comprising orally administering said composition to a human in need thereof, wherein said composition comprises an amino acid or peptide covalently attached to hydrocodone such that the pharmacological activity of hydrocodone is substantially decreased when the composition is used in a manner inconsistent with the manufacturer's instructions.

Another embodiment is a method of preventing overdose of a pharmaceutical composition, comprising orally administering said pharmaceutical composition to a human in need thereof, wherein said composition comprises a carrier peptide covalently attached to hydrocodone in a manner that substantially decreases the potential of hydrocodone to result in overdose.

Another embodiment is a method for reducing or preventing the euphoric effect of a pharmaceutical composition, comprising orally administering said composition to a human in need thereof, wherein said composition comprises a carrier peptide covalently attached to hydrocodone such that the pharmacological activity of hydrocodone is substantially decreased when the composition is used in a manner inconsistent with the manufacturer's instructions.

For each of the recited methods of the invention the following properties may be achieved through bonding hydrocodone to the chemical moiety. In one embodiment, the toxicity of the compound may be substantially lower than that of the hydrocodone when delivered in its unbound state or as a salt thereof. In another embodiment, the possibility of overdose by oral administration is reduced or eliminated. In another embodiment, the possibility of overdose by intranasal administration is reduced or eliminated. In another embodiment, the possibility of overdose by injection administration is reduced or eliminated.

Another embodiment of the invention is wherein said attachment comprises an ester or carbonate bond. Another embodiment of the invention is wherein said hydrocodone covalently attaches to a chemical moiety through a ketone and/or hydroxyl.

The compositions and methods of the invention provide hydrocodone, which when bound to the chemical moiety provide safer and/or more effective dosages for hydrocodone through improved bioavailability curves and/or safer Cₘₐₓ and/or reduce area under the curve for bioavailability, particularly for abused substances taken in doses above therapeutic levels. As a result, the compositions and methods of the invention may provide improved methods of treatment for analgesia.

Preferably, the hydrocodone prodrug exhibits an oral bioavailability of hydrocodone of at least about 60% AUC (area under the curve), more preferably at least about 70%, 80%, 90%, 95%, 96%, 97%, 98%, 99%, compared to unbound hydrocodone. Preferably, the hydrocodone prodrug exhibits a parenteral bioavailability, e.g., intranasal, bioavailability of less than about 70% AUC, more preferably less than about 50%, 30%, 20%, 15%, 10%, 5%, 4%, 3%, 2%, 1%, compared to unbound hydrocodone.

In one embodiment, the hydrocodone prodrug provides pharmacological parameters (AUC, Cₘₐₓ, Tₘₐₓ, Cₘᵢₙ, and/or t_{1/2}) within 80% to 125%, 80% to 120%, 85% to 125%, 90% to 110%, or increments therein of unbound hydrocodone. It should be recognized that the ranges can, but need not be symmetrical, e.g., 85% to 105%.

In another embodiment, the toxicity of the hydrocodone prodrug is substantially lower than that of the unbound hydrocodone. For example, in a preferred embodiment, the acute toxicity is 1-fold, 2-fold, 3-fold, 4-fold, 5-fold, 6-fold, 7-fold, 8-fold, 9-fold, 10-fold less, or increments therein less lethal than oral administration of unbound hydrocodone.

For each of the described embodiments one or more characteristics as described throughout the specification may be realized. It should also be recognized that the compounds and compositions described throughout the specification may be utilized for a variety of novel methods of treatment, reduction of abuse potential, reduction of toxicity, improved release profiles, etc. An embodiment may obtain, one or more of: a conjugate with toxicity of hydrocodone that is substantially lower than that of unbound hydrocodone; a conjugate where the covalently bound chemical moiety reduces or eliminates the possibility of overdose by oral administration; a conjugate where the covalently bound chemical moiety reduces or eliminates the possibility of overdose by intranasal administration; and/or a conjugate where the covalently bound chemical moiety reduces or eliminates the possibility of overdose by injection.

In accordance with the invention and as used herein, the following terms are defined with the following meanings, unless explicitly stated otherwise.

The compounds, compositions and methods of the invention utilize “hydrocodone conjugates,” which are also referred to as hydrocodone prodrugs.

Throughout this application the use of “chemical moiety”—sometimes referred to as the “conjugate” or the “carrier”—is meant to include any chemical substance, naturally occurring or synthetic that decreases the pharmacological activity until the hydrocodone is released including at least carrier peptides, glycopeptides, carbohydrates, lipids, nucleic acids, nucleosides, or vitamins. Preferably, the chemical moiety is generally recognized as safe (“GRAS”).

Throughout this application the use of “carrier peptide” is meant to include naturally occurring amino acids, synthetic amino acids, and combinations thereof. In particular, carrier peptide is meant to include at least a single amino acid, a dipeptide, a tripeptide, an oligopeptide, a polypeptide, or the nucleic acid-amino acids peptides. The carrier peptide can comprise a homopolymer or heteropolymer of naturally occurring or synthetic amino acids.

The use of the term “straight carrier peptide” is meant to include amino acids that are linked via a —C(O)—NH— linkage, also referred to herein as a “peptide bond,” but may be substituted along the side chains of the carrier peptide. Amino acids that are not joined together via a peptide bond or are not exclusively joined through peptide bonds are not meant to fall within the definition of straight carrier peptide.

The use of the term “unsubstituted carrier peptide” is meant to include amino acids that are linked via a —C(O)—NH— linkage, and are not otherwise substituted along the side chains of the carrier peptide. Amino acids that are not joined together via a peptide bond or are not exclusively joined through peptide bonds are not meant to fall within the definition of unsubstituted carrier peptide.

“Oligopeptide” is meant to include from 2 amino acids to 10 amino acids. “Polypeptides” are meant to include from 2 to 50 amino acids.

“Carbohydrates” includes sugars, starches, cellulose, and related compounds. More specific examples include for instance, fructose, glucose, lactose, maltose, sucrose, glyceraldehyde, dihydroxyacetone, erythrose, ribose, ribulose, xylulose, galactose, mannose, sedoheptulose, neuraminic acid, dextrin, and glycogen.

A “glycoprotein” is a compound containing carbohydrate (or glycan) covalently linked to protein. The carbohydrate may be in the form of a monosaccharide, disaccharide(s), oligosaccharide(s), polysaccharide(s), or their derivatives (e.g. sulfo- or phospho-substituted).

A “glycopeptide” is a compound consisting of carbohydrate linked to an oligopeptide composed of L- and/or D-amino acids. A glyco-amino-acid is a saccharide attached to a single amino acid by any kind of covalent bond. A glycosyl-amino-acid is a compound consisting of saccharide linked through a glycosyl linkage (O—, N— or S—) to an amino acid.

The “carrier range” or “carrier size” is determined based on the effect desired. It is preferably between one to 12 chemical moieties with one to 8 moieties being preferred. In another embodiment the number of chemical moieties attached is a specific number e.g., 1, 2, 3, 4, 5, 6, 7, 8, 9, or 10, etc. Alternatively, the chemical moiety may be described based on its molecular weight. It is preferred that the conjugate weight is below about 2,500 kD, more preferably below about 1,500 kD.

A “composition” as used herein, refers broadly to any composition containing a hydrocodone conjugate. A “pharmaceutical composition” refers to any composition containing a hydrocodone conjugate that only comprises components that are acceptable for pharmaceutical uses, e.g., excludes hydrocodone conjugates for immunological purposes.

Use of phrases such as “decreased”, “reduced”, “diminished”, or “lowered” includes at least a 10% change in pharmacological activity with respect to at least one

ADME characteristic or at least one of AUC, Cₘₐₓ, Tₘₐₓ, Cₘᵢₙ, and t_{1/2} with greater percentage changes being preferred for reduction in abuse potential and overdose potential. For instance, the change may also be greater than 25%, 35%, 45%, 55%, 65%, 75%, 85%, 95%, 96%, 97%, 98%, 99%, or other increments.

Use of the phrase “similar pharmacological activity” means that two compounds exhibit curves that have substantially the same AUC, Cₘₐₓ, Tₘₐₓ, Cₘᵢₙ, and/or t_{1/2} parameters, preferably within about 30% of each other, more preferably within about 25%, 20%, 10%, 5%, 2%, 1%, or other increments.

“Cₘₐₓ” is defined as the maximum concentration of free hydrocodone in the body obtained during the dosing interval.

“Tₘₐₓ” is defined as the time to maximum concentration.

“Cₘᵢₙ” is defined as the minimum concentration of hydrocodone in the body after dosing.

“t_{1/2}” is defined as the time required for the amount of hydrocodone in the body to be reduced to one half of its value.

Throughout this application, the term “increment” is used to define a numerical value in varying degrees of precision, e.g., to the nearest 10, 1, 0.1, 0.01, etc. The increment can be rounded to any measurable degree of precision. For example, the range 1 to 100 or increments therein includes ranges such as 20 to 80, 5 to 50, 0.4 to 98, and 0.04 to 98.05.

“Acute pain” is defined as sharp or severe pain or discomfort that lasts for a short period of time. Preferably, a short period of time is less than 3 months for nociceptive or neurogenic pain, and less than 6 months for psychogenic pain.

“Chronic pain” is defined as moderate to severe pain that lasts for a long period of time. Preferably, a long period of time is more than 3 months for nociceptive or neurogenic pain and more than 6 months for psychogenic pain.

Patient” as used herein, refers broadly to any animal that is in need of treatment, most preferably and animal that is in pain. The patient may be a clinical patient such as a human or a veterinary patient such as a companion, domesticated, livestock, exotic, or zoo animal. Animals may be mammals, reptiles, birds, amphibians, or invertebrates.

“Mammal” as used herein, refers broadly to any and all warm-blooded vertebrate animals of the class Mammalia, including humans, non-human primates, felines, canines, pigs, horses, sheep, etc.

“Pretreatment” as used herein, refers broadly to any and all preparation, treatment, or protocol that takes place before receiving a hydrocodone compound or composition of the invention.

“Treating” or “treatment” as used herein, refers broadly to preventing the disease, i.e., causing the clinical symptoms of the disease not to develop in a patient that may be exposed to or predisposed to the disease but does not yet experience or display symptoms of the disease, inhibiting the disease, i.e., arresting or reducing the development of the disease or its clinical symptoms, and/or relieving the disease, i.e., causing regression of the disease or its clinical symptoms. Treatment also encompasses an alleviation of signs and/or symptoms.

“Therapeutically effective amount” as used herein, refers broadly to the amount of a compound that, when administered to a patient for treating pain is sufficient to effect such treatment for pain. The “therapeutically effective amount” will vary depending on the compound, the disease and its severity and the age, weight, etc., of the patient to be treated. “Effective dosage” or “Effective amount” of the hydrocodone compound or composition is that which is necessary to treat or provide prophylaxis for hydrocodone.

“Selection of patients” and “Screening of patients” as used herein, refers broadly to the practice of selecting appropriate patients to receive the treatments described herein. Various factors including but not limited to age, weight, heath history, medications, surgeries, injuries, conditions, illnesses, diseases, infections, gender, ethnicity, genetic markers, polymorphisms, skin color, and sensitivity to hydrocodone treatment. Still other factors include those used by physicians to determine if a patient is appropriate to receive the treatments described herein.

“Diagnosis” as used herein, refers broadly to the practice of testing, assessing, assaying, and determining whether or not a patient is in pain.

Regarding stereochemistry, this patent is meant to cover all compounds discussed regardless of absolute configurations. Thus, natural, L-amino acids are discussed but the use of D-amino acids are also included, but not preferred.

For each of the embodiments recited herein, the carrier peptide may comprise of one or more of the naturally occurring (L-) amino acids: alanine, arginine, asparagine, aspartic acid, cysteine, glycine, glutamic acid, glutamine, histidine, isoleucine, leucine, lysine, methionine, proline, phenylalanine, serine, tryptophan, threonine, tyrosine, and valine. Other preferred amino acids include beta-alanine, beta-leucine, and tertiary leucine. In another embodiment the amino acid or peptide is comprised of one or more of the D-form of the naturally occurring amino acids. In another embodiment the amino acid or peptide is comprised of one or more unnatural, non-standard or synthetic amino acids such as, aminohexanoic acid, biphenylalanine, cyclohexylalanine, cyclohexylglycine, diethylglycine, dipropylglycine, 2,3-diaminoproprionic acid, homophenylalanine, homoserine, homotyrosine, naphthylalanine, norleucine, ornithine, phenylalanine(4-fluoro), phenylalanine(2,3,4,5,6 pentafluoro), phenylalanine(4-nitro), phenylglycine, pipecolic acid, sarcosine, tetrahydroisoquinoline-3-carboxylic acid, and tert-leucine. In another embodiment the amino acid or peptide comprises of one or more amino acid alcohols. In another embodiment the amino acid or peptide comprises of one or more N-methyl amino acids.

In another embodiment, the specific carriers listed in the table may have one or more of amino acids substituted with one of the 20 naturally occurring amino acids. It is preferred that the substitution be with an amino acid which is similar in structure or charge compared to the amino acid in the sequence. For instance, isoleucine (Ile)[I] is structurally very similar to leucine (Leu)[L], whereas, tyrosine (Tyr)[Y] is similar to phenylalanine (Phe)[F], whereas serine (Ser)[S] is similar to threonine (Thr)[T], whereas cysteine (Cys)[C] is similar to methionine (Met)[M], whereas alanine (Ala)[A] is similar to valine (Val) [V], whereas lysine (Lys)[K] is similar to arginine (Arg)[R], whereas asparagine (Asn)[N] is similar to glutamine (Gln)[Q], whereas aspartic acid (Asp)[D] is similar to glutamic acid (Glu)[E], whereas histidine (His)[H] is similar to proline (Pro)[P], and glycine (Gly)[G] is similar to tryptophan (Trp)[W]. In the alternative the preferred amino acid substitutions may be selected according to hydrophilic properties (i.e., polarity) or other common characteristics associated with the 20 essential amino acids. While preferred embodiments utilize the 20 natural amino acids for their GRAS characteristics, it is recognized that minor substitutions along the amino acid chain that do not affect the essential characteristics of the amino are also contemplated.

The hydrocodone conjugate may also be in salt form. Pharmaceutically acceptable salts, e.g., non-toxic, inorganic and organic acid addition salts, are known in the art. Exemplary salts include, but are not limited to, 2-hydroxyethanesulfonate, 2-naphthalenesulfonate, 3-hydroxy-2-naphthoate, 3-phenylpropionate, acetate, adipate, alginate, amsonate, aspartate, benzenesulfonate, benzoate, bisulfate, bitartrate, borate, butyrate, calcium edetate, camphorate, camphorsulfonate, citrate, clavulariate, cyclopentanepropionate, digluconate, dodecylsulfate, edetate, edisylate, estolate, esylate, ethanesulfonate, fumarate, gluceptate, glucoheptanoate, gluconate, glutamate, glycerophosphate, glycollylarsanilate, hemisulfate, heptanoate, hexafluorophosphate, hexanoate, hexylresorcinate, hydrabamine, hydrobromide, hydrochloride, hydroiodide, hydroxynaphthoate, isothionate, lactate, lactobionate, laurate, laurylsulphonate, malate, maleate, mandelate, methanesulfonate, mucate, naphthylate, napsylate, nicotinate, N-methylglucamine ammonium salt, oleate, palmitate, pamoate, pantothenate, pectinate, phosphate, phosphateldiphosphate, pivalate, polygalacturonate, propionate, p-toluenesulfonate, saccharate, salicylate, stearate, subacetate, succinate, sulfate, sulfosaliculate, suramate, tannate, tartrate, teoclate, tosylate, triethiodide, undecanoate, and valerate salts, and the like.

In the invention, hydrocodone may be covalently attached to the peptide via the ketone group and a linker. This linker may be a small linear or cyclic molecule containing 2-6 atoms with one or more heteroatoms (such as O, S, N) and one or more functional groups (such as amines, amides, alcohols or acids) or may be made up of a short chain of either amino acids or carbohydrates). For example, glucose would be suitable as a linker.

In yet another embodiment of the invention, linkers can be selected from the group of all chemical classes of compounds such that virtually any side chain of the peptide can be attached. The linker should have a functional pendant group, such as a carboxylate, an alcohol, thiol, oxime, hydraxone, hydrazide, or an amine group, to covalently attach to the carrier peptide. In one preferred embodiment, the alcohol group of hydrocodone is covalently attached to the N-terminus of the peptide via a linker. In another preferred embodiment the ketone group of hydrocodone is attached to a linker through the formation of a ketal and the linker has a pendant group that is attached to the carrier peptide.

Additionally information regarding the attachment of active agents such as hydrocodone to carriers may be found in U.S. Pat. No. 7,060,708 and/or PCT/US03/05524 (WO 03/079972 A1), and/or PCT/US03/05525 (WO 03/072046 A1), and/or U.S. Patent Application Publication US 2005/0266070 A1 each of which is hereby incorporated by reference in its entirety.

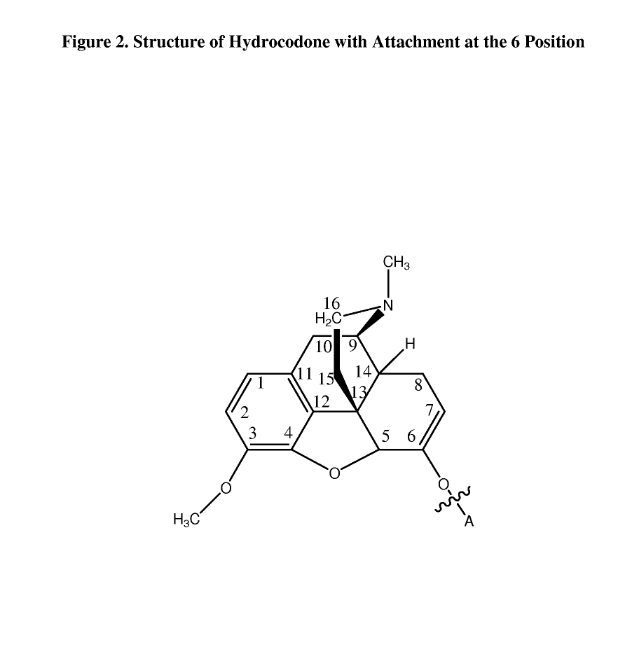

Referring now to FIG. 2, this Figure shows the potential attachment sites of hydrocodone. Specifically, hydrocodone may be attached to the chemical moiety at the 6 positions.

In addition to the hydrocodone prodrug, the pharmaceutical compositions of the invention may further comprise one or more pharmaceutical additives. Pharmaceutical additives include a wide range of materials including, but not limited to diluents and bulking substances, binders and adhesives, lubricants, glidants, plasticizers, disintegrants, carrier solvents, buffers, colorants, flavorings, sweeteners, preservatives and stabilizers, adsorbents, and other pharmaceutical additives known in the art.

Lubricants include, but are not limited to, magnesium stearate, calcium stearate, zinc stearate, powdered stearic acid, glyceryl monostearate, glyceryl palmitostearate, glyceryl behenate, silica, magnesium silicate, colloidal silicon dioxide, titanium dioxide, sodium benzoate, sodium lauryl sulfate, sodium stearyl fumarate, hydrogenated vegetable oil, talc, polyethylene glycol, and mineral oil.

Surface agents for formulation include, but are not limited to, sodium lauryl sulfate, dioctyl sodium sulfosuccinate, triethanolamine, polyoxyethylene sorbitan, poloxalkol, and quarternary ammonium salts; excipients such as lactose, mannitol, glucose, fructose, xylose, galactose, sucrose, maltose, xylitol, sorbitol, chloride, sulfate and phosphate salts of potassium, sodium, and magnesium; gelling agents such as colloidal clays; thickening agents such as gum tragacanth or sodium alginate, effervescing mixtures; and wetting agents such as lecithin, polysorbates or laurylsulphates.

Colorants can be used to improve appearance or to help identify the pharmaceutical composition. See 21 C.F.R., Part 74. Exemplary colorants include D&C Red No. 28, D&C Yellow No. 10, FD&C Blue No. 1, FD&C Red No. 40, FD&C Green #3, FD&C Yellow No. 6, and edible inks.

In embodiments where the pharmaceutical composition is compacted into a solid dosage form, e.g., a tablet, a binder can help the ingredients hold together. Binders include, but are not limited to, sugars such as sucrose, lactose, and glucose; corn syrup; soy polysaccharide, gelatin; povidone (e.g., Kollidon®, Plasdone®); Pullulan; cellulose derivatives such as microcrystalline cellulose, hydroxypropylmethyl cellulose (e.g., Methocel®), hydroxypropyl cellulose (e.g., Klucel®), ethylcellulose, hydroxyethyl cellulose, carboxymethylcellulose sodium, and methylcellulose; acrylic and methacrylic acid co-polymers; carbomer (e.g., Carbopol®); polyvinylpolypyrrolidine, polyethylene glycol (Carbowax®); pharmaceutical glaze; alginates such as alginic acid and sodium alginate; gums such as acacia, guar gum, and arabic gums; tragacanth; dextrin and maltodextrin; milk derivatives such as whey; starches such as pregelatinized starch and starch paste; hydrogenated vegetable oil; and magnesium aluminum silicate, as well as other conventional binders known to persons skilled in the art. Exemplary non-limiting bulking substances include sugar, lactose, gelatin, starch, and silicon dioxide.

Glidants can improve the flowability of non-compacted solid dosage forms and can improve the accuracy of dosing. Glidants include, but are not limited to, colloidal silicon dioxide, fumed silicon dioxide, silica gel, talc, magnesium trisilicate, magnesium or calcium stearate, powdered cellulose, starch, and tribasic calcium phosphate.

Plasticizers include, but are not limited to, hydrophobic and/or hydrophilic plasticizers such as, diethyl phthalate, butyl phthalate, diethyl sebacate, dibutyl sebacate, triethyl citrate, acetyltriethyl citrate, acetyltributyl citrate, cronotic acid, propylene glycol, castor oil, triacetin, polyethylene glycol, propylene glycol, glycerin, and sorbitol. Plasticizers are particularly useful for pharmaceutical compositions containing a polymer and in soft capsules and film-coated tablets.

Flavorings improve palatability and may be particularly useful for chewable tablet or liquid dosage forms. Flavorings include, but are not limited to maltol, vanillin, ethyl vanillin, menthol, citric acid, fumaric acid, ethyl maltol, and tartaric acid. Sweeteners include, but are not limited to, sorbitol, saccharin, sodium saccharin, sucrose, aspartame, fructose, mannitol, and invert sugar.

Preservatives and/or stabilizers improving the ability to store the compositions include, but are not limited to, alcohol, sodium benzoate, butylated hydroxy toluene, butylated hydroxyanisole, and ethylenediamine tetraacetic acid.

Disintegrants can increase the dissolution rate of a pharmaceutical composition. Disintegrants include, but are not limited to, alginates such as alginic acid and sodium alginate, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), polyvinylpolypyrrolidine (Plasone-XL®), guar gum, magnesium aluminum silicate, methyl cellulose, microcrystalline cellulose, polacrilin potassium, powdered cellulose, starch, pregelatinized starch, sodium starch glycolate (e.g., Explotab®, Primogel®).

Diluents increase the bulk of a dosage form and may make the dosage form easier to handle. Exemplary diluents include, but are not limited to, lactose, dextrose, saccharose, cellulose, starch, and calcium phosphate for solid dosage forms, e.g., tablets and capsules; olive oil and ethyl oleate for soft capsules; water and vegetable oil for liquid dosage forms, e.g., suspensions and emulsions. Additional suitable diluents include, but are not limited to, sucrose, dextrates, dextrin, maltodextrin, microcrystalline cellulose (e.g., Avicel®), microfine cellulose, powdered cellulose, pregelatinized starch (e.g., Starch 1500®), calcium phosphate dihydrate, soy polysaccharide (e.g., Emcosoy®), gelatin, silicon dioxide, calcium sulfate, calcium carbonate, magnesium carbonate, magnesium oxide, sorbitol, mannitol, kaolin, polymethacrylates (e.g., Eudragit®), potassium chloride, sodium chloride, and talc.

In embodiments where the pharmaceutical composition is formulated for a liquid dosage form, the pharmaceutical composition may include one or more solvents. Suitable solvents include, but are not limited to, water; alcohols such as ethanol and isopropyl alcohol; vegetable oil; polyethylene glycol; propylene glycol; and glycerin or mixing and combination thereof.

The pharmaceutical composition can comprise a buffer. Buffers include, but are not limited to, lactic acid, citric acid, acetic acid, sodium lactate, sodium citrate, and sodium acetate.

Hydrophilic polymers suitable for use in the sustained release formulation include: one or more natural or partially or totally synthetic hydrophilic gums such as acacia, gum tragacanth, locust bean gum, guar gum, or karaya gum, modified cellulosic substances such as methylcellulose, hydroxymethylcellulose, hydroxypropyl methylcellulose, hydroxypropyl cellulose, hydroxyethylcellulose, carboxymethylcellulose; proteinaceous substances such as agar, pectin, carrageen, and alginates; and other hydrophilic polymers such as carboxypolymethylene, gelatin, casein, zein, bentonite, magnesium aluminum silicate, polysaccharides, modified starch derivatives, and other hydrophilic polymers known to those of skill in the art or a combination of such polymers.

One of ordinary skill in the art would recognize a variety of structures, such as bead constructions and coatings, useful for achieving particular release profiles. It is also possible for the dosage form to combine any forms of release known to persons of ordinary skill in the art. These include immediate release, extended release, pulse release, variable release, controlled release, timed release, sustained release, delayed release, long acting, and combinations thereof. The ability to obtain immediate release, extended release, pulse release, variable release, controlled release, timed release, sustained release, delayed release, long acting characteristics and combinations thereof is known in the art. See, e.g., U.S. Pat. No. 6,913,768.

However, it should be noted that the hydrocodone conjugate controls the release of hydrocodone into the digestive tract over an extended period of time resulting in an improved profile when compared to immediate release combinations and reduces and/or prevents abuse without the addition of the above additives. In a preferred embodiment no further sustained release additives are required to achieve a blunted or reduced pharmacokinetic curve (e.g. reduced euphoric effect) while achieving therapeutically effective amounts of hydrocodone release.

The dose range for adult human beings will depend on a number of factors including the age, weight and condition of the patient and the administration route. Tablets and other forms of presentation provided in discrete units conveniently contain a daily dose, or an appropriate fraction thereof, of the hydrocodone conjugate. The dosage form can contain a dose of about 2.5 mg to about 500 mg, about 10 mg to about 250 mg, about 10 mg to about 100 mg, about 25 mg to about 75 mg, or increments therein. In a preferred embodiment, the dosage form contains 5 mg, 7.5 mg, 10 mg, 12 mg, 18 mg, 24 mg, 30 mg, or 50 mg of a hydrocodone prodrug.

Tablets and other dosage forms provided in discrete units can contain a daily dose, or an appropriate fraction thereof, of one or more hydrocodone prodrugs.

Compositions of the invention may be administered in a partial, i.e., fractional dose, one or more times during a 24 hour period, a single dose during a 24 hour period of time, a double dose during a 24 hour period of time, or more than a double dose during a 24 hour period of time. Fractional, double or other multiple doses may be taken simultaneously or at different times during the 24-hour period. The doses may be uneven doses with regard to one another or with regard to the individual components at different administration times. Preferably, a single dose is administered once daily.

Likewise, the compositions of the invention may be provided in a blister pack or other such pharmaceutical package. Further, the compositions of the present inventive subject matter may further include or be accompanied by indicia allowing individuals to identify the compositions as products for a prescribed treatment. The indicia may further additionally include an indication of the above specified time periods for administering the compositions. For example the indicia may be time indicia indicating a specific or general time of day for administration of the composition, or the indicia may be a day indicia indicating a day of the week for administration of the composition. The blister pack or other combination package may also include a second pharmaceutical product.

The compounds of the invention can be administered by a variety of dosage forms. Any biologically acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspension in an aqueous liquid or a non-aqueous liquid, emulsions, tablets, syringes, multi-layer tablets, bi-layer tablets, capsules, soft gelatin capsules, hard gelatin capsules, caplets, lozenges, chewable lozenges, beads, powders, granules, particles, microparticles, dispersible granules, cachets, and combinations thereof. Preferably, said composition may be in the form of any of the known varieties of tablets (e.g., chewable tablets, conventional tablets, film-coated tablets, compressed tablets), capsules, liquid dispersions for oral administration (e.g., syrups, emulsions, solutions or suspensions).

However, the most effective means for delivering the abuse-resistant hydrocodone compounds of the invention is orally, to permit maximum release of hydrocodone to provide therapeutic effectiveness and/or sustained release while maintaining abuse resistance. When delivered by the oral route hydrocodone is released into circulation, preferably over an extended period of time as compared to hydrocodone alone.

It is preferred that the hydrocodone conjugate be compact enough to allow for a reduction in overall administration size. The smaller size of the hydrocodone prodrug dosage forms promotes ease of swallowing.

For oral administration, fine powders or granules containing diluting, dispersing and/or surface-active agents may be presented in a draught, in water or a syrup, in capsules or sachets in the dry state, in a non-aqueous suspension wherein suspending agents may be included, or in a suspension in water or a syrup. Where desirable or necessary, flavoring, preserving, suspending, thickening or emulsifying agents can be included.

Accordingly, the invention also provides methods comprising providing, administering, prescribing, or consuming a hydrocodone prodrug. The invention also provides pharmaceutical compositions comprising a hydrocodone prodrug. The formulation of such a pharmaceutical composition can optionally enhance or achieve the desired release profile.

Any feature of the above-describe embodiments can be used in combination with any other feature of the above-described embodiments.

In order to facilitate a more complete understanding of the invention, Examples are provided below. However, the scope of the invention is not limited to specific embodiments disclosed in these Examples, which are for purposes of illustration only.

The following Table lists carrier peptides to which hydrocodone may be covalently bonded.

TABLE 1

List of Prefeffed Amino Acids and Peptides to which
Hydrocodone May be Covalently Bonded

| | | | |
|---|---|---|---|
| Ala | Glu-Val-Val | Phe-Ser-Val | Tyr-Tyr-Phe |
| Arg | Gly-Asp-Val | Phe-Thr-Val | Tyr-Tyr-Val |
| Asn | Gly-Gly-Cha | Phe-Tyr-Val | Tyr-Val-Val |
| Asp | Gly-Gly-Phe | Pro-Asp-Val | Val-Asp-Val |
| Cys | Gly-Gly-Ile | Pro-Gly-Val | Val-Gln-Val |
| Gln | Gly-Gly-Leu | Pro-Ile-Ile | Val-Glu-Gly |
| Glu | Gly-Pro-Val | Pro-Ile-Val | Val-Glu-Leu |
| Gly | Gly-Ser-Val | Pro-Leu-Ile | Val-Glu-Val |
| His | Gly-Thr-Val | Pro-Lys-Val | Val-Gly-Glu |
| Ile | Gly-Val-Val | Pro-Phe-Ile | Val-Gly-Val |
| Leu | Gly-Gly-Nle | Pro-Phe-Val | Val-Phe-Val |
| Lys | Gly-Gly-Phe | Pro-Pro-Cha | Val-Pro-Tyr |
| Met | Gly-Gly-Val | Pro-Pro-Ile | Val-Pro-Val |
| Phe | Gly-Ile-Ile | Pro-Pro-Leu | Val-Thr-Val |
| Pro | Gly-Lys-Val | Pro-Pro-Nle | Val-Tyr-Asp |
| Ser | Ile-Asp-Val | Pro-Pro-Phe | Val-Tyr-Asp |
| β-Leu | Ile-Glu-Val | Pro-Pro-Val | Val-Tyr-Glu |
| Thr | Ile-Gly-Val | Pro-Pro-Val | Val-Tyr-Gly |
| t-Leu | Ile-Phe-Val | Pro-Ser-Val | Val-Tyr-Ile |
| Trp | Ile-Ser-Val | Pro-Thr-Val | Val-Tyr-Leu |
| Tyr | Ile-Thr-Val | Pro-Tyr-Val | Val-Tyr-Lys |
| Val | Ile-Tyr-Val | Pro-Tyr-Val | Val-Tyr-Phe |
| β-Ala | Leu-Asp-Val | Pro-Val-Val | Val-Tyr-Pro |
| Glu_{pyro}-Glu | Leu-Glu-Val | Ser-Asp-Val | Val-Tyr-Val |
| Tyr-β-Ala | Leu-Gly-Val | Ser-Glu-Val | Lys-Tyr-Val-Ile |
| | | | [SEQ ID NO: 1] |
| β-Ala-β-Ala | Leu-Leu-Ile | Ser-Gly-Val | Tyr-Pro-Val-Ile |
| | | | [SEQ ID NO: 2] |
| Asp-Asp-Cha | Leu-Lys-Val | Ser-Ile-Val | Acetyl-Glu-Glu-Pro-Pro-Ile |
| | | | [SEQ ID NO: 3] |
| Asp-Asp-Ile | Leu-Phe-Val | Ser-Leu-Val | Asp-Asp-Gly-Gly-Ile |
| | | | [SEQ ID NO: 4] |
| Asp-Asp-Nle | Leu-Pro-Ile | Ser-Lys-Val | Asp-Asp-Leu-Leu-Ile |
| | | | [SEQ ID NO: 5] |
| Asp-Asp-Phe | Leu-Pro-Val | Ser-Phe-Val | Asp-Asp-Leu-Leu-Ile |
| | | | [SEQ ID NO: 6] |
| Asp-Asp-Val | Leu-Thr-Val | Ser-Pro-Val | Asp-Asp-Pro-Pro-Ile |
| | | | [SEQ ID NO: 7] |
| Asp-d-Asp-Ile | Leu-Tyr-Val | Ser-Tyr-Val | Glu-Glu-Gly-Gly-Phe |
| | | | [SEQ ID NO: 8] |
| Asp-Glu-Val | Lys-Asp-Val | Ser-Val-Val | Glu-Glu-Leu-Leu-Leu |
| | | | [SEQ ID NO: 9] |
| Asp-Gly-Val | Lys-Glu-Val | Thr-Asp-Val | Glu-Glu-Phe-Phe-Leu |
| | | | [SEQ ID NO: 10] |
| Asp-Ile-Val | Lys-Gly-Val | Thr-Glu-Val | Glu-Glu-Phe-Pro-Ile |
| | | | [SEQ ID NO: 11] |
| Asp-Leu-Val | Lys-Ile-Val | Thr-Gly-Val | Glu-Glu-Pro-Pro-Leu |
| | | | [SEQ ID NO: 12] |
| Asp-Lys-Val | Lys-Leu-Val | Thr-Leu-Val | Glu-Glu-Pro-Phe-Ile |
| | | | [SEQ ID NO: 13] |
| Asp-Phe-Val | Lys-Lys-Ile | Thr-Lys-Val | Glu-Glu-Glu-Glu-Ile |
| | | | [SEQ ID NO: 14] |
| Asp-Pro-Val | Lys-Lys-Leu | Thr-Phe-Val | Glu-Glu-Phe-Phe-Phe |
| | | | [SEQ ID NO: 15] |
| Asp-Ser-Val | Lys-Lys-Val | Thr-Pro-Val | Gly-Gly-Glu-Glu-Ile |
| | | | [SEQ ID NO: 16] |
| Asp-Thr-Val | Lys-Phe-Val | Thr-Ser-Val | Lys-Lys-Leu-Leu-Ile |
| | | | [SEQ ID NO: 17] |
| Asp-Tyr-Val | Lys-Pro-Val | Thr-Thr-Ile | Lys-Lys-Pro-Pro-Ile |
| | | | [SEQ ID NO: 18] |
| Asp-Val-Val | Lys-Thr-Val | Thr-Thr-Val | Phe-Phe-Glu-Glu-Ile |
| | | | [SEQ ID NO: 19] |
| Gln-Gln-Ile | Lys-Tyr-Val | Thr-Tyr-Val | Phe-Phe-Phe-Phe-Phe |
| | | | [SEQ ID NO: 20] |
| Gln-Gln-Val | Lys-Tyr-Val | Thr-Val-Val | Thr-Thr-Gly-Gly-Ile |
| | | | [SEQ ID NO: 21] |
| Gln-Gln-β-Ala | Lys-Val-Val | Tyr-Asp-Val | Thr-Thr-Phe-Phe-Ile |
| | | | [SEQ ID NO: 22] |
| Gln-Pro-Val | Phe-Asp-Val | Tyr-Glu-Val | Tyr-Tyr-Leu-Leu-Ile |
| | | | [SEQ ID NO: 23] |
| Glu-Glu-Cha | Phe-Glu-Val | Tyr-Gly-Val | Tyr-Tyr-Phe-Phe-Ile |
| | | | [SEQ ID NO: 24] |
| Glu-Glu-hPhe | Phe-Gly-Val | Tyr-Ile-Val | Tyr-Tyr-Pro-Pro-Ile |
| | | | [SEQ ID NO: 25] |
| Glu-Glu-Ile | Phe-Ile-Val | Tyr-Leu-Val | Tyr-Tyr-Pro-Phe-Ile |
| | | | [SEQ ID NO: 26] |
| Glu-Glu-Leu | Phe-Leu-Val | Tyr-Lys-Val | Tyr-Tyr-Phe-Phe-Ile |
| | | | [SEQ ID NO: 27] |
| Glu-Glu-Nle | Phe-Lys-Val | Tyr-Phe-Val | Tyr-Tyr-Phe-Phe-Val |
| | | | [SEQ ID NO: 28] |
| Glu-Glu-Phe | Phe-Phe-Cha | Tyr-Pro-Val | Asp-Asp-Lys(Asp₂) |
| Glu-Glu-Val | Phe-Phe-hPhe | Tyr-Ser-Val | Glu-Glu-Lys(Glu₂) |
| Glu-Gly-Val | Phe-Phe-Ile | Tyr-Thr-Val | Phe-Phe-Lys(Phe₂) |
| Glu-Leu-Val | Phe-Phe-Leu | Tyr-Tyr-Ala | Pro-Pro-Lys(Pro₂) |
| Glu-Lys-Val | Phe-Phe-Nle | Tyr-Tyr-Cha | Tyr-Tyr-Lys(Tyr₂) |
| Glu-Phe-Val | Phe-Phe-Phe | Tyr-Tyr-hPhe | Ethyl Carbonate |
| Glu-Ser-Val | Phe-Phe-Val | Tyr-Tyr-Ile | galactose-Gly-Gly-Ile |
| Glu-Thr-Val | Phe-Phe-Val | Tyr-Tyr-Leu | galactose-Gly-Gly-Leu |
| Glu-Tyr-Val | Phe-Pro-Val | Tyr-Tyr-Nle | galactose-Ile |

The following Table lists preferred hydrocodone conjugates made according to the invention.

TABLE 2

List of Hydrocodone (HC) Conjugates attached through the 6
position to the C-terminus of the amino acid according to the
invention (for clarity purposes the amino acid that is next to
the -HC is the amino acid that is connected to the HC).

| | | |
|---|---|---|
| Aib-HC | Phe-Phe-Ile-HC | Lys-Lys-Gly-Gly-Ile-HC |
| | | [SEQ ID NO: 29] |
| Boc-Glu(OtBu)-HC | Phe-Phe-Leu-HC | Lys-Lys-Leu-Leu-Ile-HC |
| | | [SEQ ID NO: 30] |
| Boc-Lys(Boc)-HC | Phe-Phe-Phe-HC | Lys-Lys-Pro-Pro-Ile-HC |
| | | [SEQ ID NO: 31] |
| Glu-HC | Pro-Ile-Ile-HC | Phe-Phe-Glu-Glu-Ile-HC |
| | | [SEQ ID NO: 32] |
| Gly-HC | Pro-Leu-Ile-HC | Phe-Phe-Phe-Phe-Phe-HC |
| | | [SEQ ID NO: 33] |
| Ile-HC | Pro-Phe-Ile-HC | Thr-Thr-Gly-Gly-Ile-HC |
| | | [SEQ ID NO: 34] |
| Leu-HC | Pro-Pro-Glu-HC | Thr-Thr-Phe-Phe-Ile-HC |
| | | [SEQ ID NO: 35] |
| Lys-HC | Pro-Pro-Ile-HC | Tyr-Tyr-Glu-Glu-Ile-HC |
| | | [SEQ ID NO: 36] |
| Phe-HC | Pro-Pro-Leu-HC | Tyr-Tyr-Gly-Gly-Ile-HC |
| | | [SEQ ID NO: 37] |
| Pro-HC | Pro-Pro-Phe-HC | Tyr-Tyr-Leu-Leu-Ile-HC |
| | | [SEQ ID NO: 38] |
| Ser-HC | Thr-Thr-Ile-HC | Tyr-Tyr-Phe-Pro-Ile-HC |
| | | [SEQ ID NO: 39] |
| Ala-Pro-HC | Tyr-Tyr-Ile-HC | Tyr-Tyr-Pro-Pro-Ile-HC |
| | | [SEQ ID NO: 40] |
| Boc-Ala-Pro-HC | Gln-Gln-Ile-HC | Tyr-Tyr-Pro-Phe-Ile-HC |
| | | [SEQ ID NO: 41] |
| Boc-Glu(OtBu)-Leu-HC | Gly-Gly-Gly-Gly-HC | Tyr-Tyr-Phe-Phe-Ile-HC |
| | [SEQ ID NO: 44] | [SEQ ID NO: 42] |
| Boc-Glu(OtBu)-Pro-HC | Acetyl-Glu-Glu-Pro-Pro-Ile-HC | Glu-Glu-Phe-Phe-Phe-Ile-HC |
| | [SEQ ID NO: 45] | [SEQ ID NO: 43] |
| Glu-Glu-HC | Asp-Asp-Gly-Gly-Ile-HC | β-Ala-HC |
| | [SEQ ID NO: 46] | |
| Glu-Leu-HC | Asp-Asp-Leu-Leu-Ile-HC | β-Ala-β-Ala-HC |
| | [SEQ ID NO: 47] | |
| Glu-Pro-HC | Asp-Asp-Phe-Phe-Ile-HC | EpE-HC |
| | [SEQ ID NO: 48] | |
| Glu_{pyro}-Glu-HC | Asp-Asp-Pro-Pro-Ile-HC | Ethyl Carbonate-HC |
| | [SEQ ID NO: 49] | |
| Asp-Asp-Ile-HC | Asp-Asp-Asp-Asp-Ile-HC | Galactose-CO-Leu-HC |
| | [SEQ ID NO: 50] | |
| Gln-Gln-Ile-HC | Glu-Glu-Asp-Asp-Ile-HC | Galactose-CO-Pro-Pro-Ile-HC |
| | [SEQ ID NO: 51] | |
| Glu-Glu-Glu-HC | Glu-Glu-Gly-Gly-Aib-HC | Galactose-CO-Pro-Pro-Leu-HC |
| | [SEQ ID NO: 52] | |
| Glu-Glu-Ile-HC | Glu-Glu-Gly-Gly-Ile-HC | galactose-Gly-Gly-Ile-HC |
| | [SEQ ID NO: 53] | |
| Glu-Glu-Leu-HC | Glu-Glu-Gly-Gly-Leu-HC | galactose-Gly-Gly-Leu-HC |
| | [SEQ ID NO: 54] | |
| Gly-Gly-Aib-HC | Glu-Glu-Gly-Gly-Phe-HC | galactose-Ile-HC |
| | [SEQ ID NO: 55] | |
| Gly-Gly-Glu-HC | Glu-Glu-Leu-Leu-Leu-HC | Gulonic acid-Ile-HC |
| | [SEQ ID NO: 56] | |
| Gly-Gly-Ile-HC | Glu-Glu-Phe-Phe-Leu-HC | |
| | [SEQ ID NO: 57] | |
| Gly-Gly-Leu-HC | Glu-Glu-Phe-Pro-Ile-HC | |
| | [SEQ ID NO: 58] | |
| Gly-Gly-Phe-HC | Glu-Glu-Pro-Pro-Leu-HC | |
| | [SEQ ID NO: 59] | |
| Gly-Ile-Ile-HC | Glu-Glu-Pro-Phe-Ile-HC | |
| | [SEQ ID NO: 60] | |
| Gly-Leu-Ile-HC | Glu-Glu-Glu-Glu-Ile-HC | |
| | [SEQ ID NO: 61] | |
| Gly-Leu-Leu-HC | Glu-Glu-Glu-Glu-Glu-HC | |
| | [SEQ ID NO: 62] | |
| Gly-Phe-Ile-HC | Glu-Glu-Phe-Phe-Ile-HC | |
| | [SEQ ID NO: 63] | |
| Gly-Phe-Leu-HC | Glu-Glu-Phe-Phe-Phe-HC | |
| | [SEQ ID NO: 64] | |
| Leu-Leu-Glu-HC | Gly-Gly-Glu-Glu-Ile-HC | |
| | [SEQ ID NO: 65] | |
| Leu-Leu-Ile-HC | Gly-Gly-Glu-Glu-Glu-HC | |
| | [SEQ ID NO: 66] | |
| Leu-Leu-Leu-HC | Gly-Gly-Pro-Pro-Ile-HC | |
| | [SEQ ID NO: 67] | |
| Leu-Pro-Glu-HC | Gly-Gly-Gly-Gly-Aib-HC | |
| | [SEQ ID NO: 68] | |
| Leu-Pro-Ile-HC | Gly-Gly-Gly-Gly-Ile-HC | |
| | [SEQ ID NO: 69] | |
| Leu-Pro-Leu-HC | Gly-Gly-Gly-Gly-Leu-HC | |
| | [SEQ ID NO: 70] | |
| Leu-Pro-Phe-HC | Gly-Gly-Gly-Gly-Phe-HC | |
| | [SEQ ID NO: 71] | |
| (d)-Lys-(1)-Lys-Ile-HC | Lys-Lys-Asp-Asp-Ile-HC | |
| | [SEQ ID NO: 72] | |
| Lys-Lys-Ile-HC | Lys-Lys-Glu-Glu-Ile-HC | |
| | [SEQ ID NO: 73] | |

In order to facilitate a more complete understanding of the invention, Examples are provided below. However, the scope of the invention is not limited to specific embodiments disclosed in these Examples, which are for purposes of illustration only.

### EXAMPLES

The Examples illustrate the applicability of attaching various moieties to hydrocodone to reduce the potential for overdose while maintaining therapeutic value. The invention is illustrated by pharmacokinetic studies with various peptide opioid (e.g. hydrocodone) conjugates. The pharmacokinetics of the parent opioid (e.g. hydrocodone) and major active metabolites (e.g. hydromorphone and oxymorphone) following oral, intravenous, or intranasal administration of the peptide-opioid conjugate or the parent drug at equimolar amounts were determined in rats.

Oral, intranasal, and intravenous bioavailability studies of hydrocodone and hydrocodone conjugates were conducted in male Sprague-Dawley rats. Doses of hydrocodone bitartrate and hydrocodone conjugates containing equivalent amounts of hydrocodone were administered in deionized water. Oral administration was in 0.5 ml by gavage needle (with the exception of YYI-HC, which was delivered as a solid in gelatin capsules). Intranasal doses were administered by placing 20 microliters into the nasal flares of rats anesthetized with isoflurane. Intravenous administration was in 0.1 ml by tail vein injection. Plasma was collected by retroorbital sinus puncture under isoflurane anesthesia. Hydrocodone and hydromorphone (major active metabolite) concentrations were determined by LC/MS/MS.

The below examples are illustrative only and the below amino acid sequences attached to hydrocodone is not meant to be limiting. As such, synthesis and attachment of hydrocodone may be accomplished for instance view the following exemplary methods.

Peptide conjugates were synthesized by the general method described in below.

Hydrocodone free base was treated with a base (LHMTS, K-t-BuO, Li-t-BuO) followed by addition of N-protected activated amino acid. The product then obtained was nitrogen deprotected to yield an amino-acid linked hydrocodone.

An iterative approach can be used to identify favorable conjugates by synthesizing and testing single amino acid conjugates, and then extending the peptide one amino acid at a time to yield dipeptide and tripeptide conjugates, etc. The parent single amino acid prodrug candidate may exhibit more or less desirable characteristics than its di- or tripeptide offspring candidates.

Mono-Substituted Hydrocodone Conjugates

Single Amino Acid Hydrocodone Conjugates

### Example 1

### Leu-Hydrocodone

| | | | | | | Molar |
|---|---|---|---|---|---|---|
| Reagents | | MW | Weight | | mmoles | Equivalents |
| 1. | Hydrocodone | 299 | 1.00 | g | 3.34 | 1.0 |
| 1. | LiN(TMS)₂ in THF | 1M | 10.5 | ml | 10.5 | 3.15 |
| 1. | THF | — | 25 | ml | — | — |
| 2. | Boc-Leu-OSu | 328 | 3.28 | g | 10.0 | 3.0 |

To a solution of hydrocodone in THF was added LiN(TMS)₂ in THF via syringe. The solution was stirred at ambient temperatures for 5 minutes then Boc-Leu-OSu was added. The resulting reaction mixture was stirred at ambient temperatures for 18 hours. Reaction was neutralized to pH 7 with 6M HCl. Solvent was removed. Crude material was taken up in CHCl₃ (100 ml), washed with sat. NaHCO₃ (3×100 ml), dried over MgSO₄, filtered, and solvent removed. Solid was collected as a yellow powder (1.98 g, 95% yield): ¹H NMR (DMSO-d₆) δ 0.86 (dd, 6H), 1.31 (s, 9H), 1.46 (s, 2H), 1.55 (m, 2H), 1.69 (m, 1H), 1.87 (dt, 1H), 2.07 (dt, 2H), 2.29 (s, 3H), 2.43 (m, 2H), 2.93 (d, 1H), 3.11 (s, 1H), 3.72 (s, 3H), 3.88 (dt, 1H), 4.03 (dt, 1H), 4.87 (s, 1H), 5.51 (d, 1H), 6.65 (d, 1H), 6.73 (d, 1H), 6.90 (s, 1H).

To the Boc-Leu-Hydrocodone was added 25 ml of 4N HCl in dioxane. The resulting mixture was stirred at ambient temperatures for 18 hours. Solvent was removed and final product dried under vacuum. Solid was collected as a slightly yellow solid (1.96 g, 97% yield): ¹H NMR (DMSO-d₆) δ 0.94 (d, 6H), 1.52 (m, 1H), 1.75-1.90 (m, 4H), 2.22 (dt, 1H), 2.34 (dt, 1H), 2.64 (q, 1H), 2.75 (s, 3H), 2.95-3.23 (m, 4H), 3.74 (s, 3H), 3.91 (d, 1H), 4.07 (s, 1H), 5.10 (s, 1H), 5.72 (d, 1H), 6.76 (d, 1H), 6.86 (d, 1H), 8.73 br s, 3H).

Dipeptide Hydrocodone Conjugates

### Example 2

### Example of Conjugates Containing Two Different Amino Acids: Ala-Pro-Hydrocodone

| Reagents | MW | Weight | | mmoles | Molar Equivalents |
|---|---|---|---|---|---|
| Pro-Hydrocodone | 468 | 0.25 | g | 0.53 | 1.0 |
| Boc-Ala-OSu | 286 | 0.33 | g | 1.2 | 2.26 |
| NMM | 101 | 0.50 | ml | 5.38 | 10.2 |
| DMF | — | 10 | ml | — | — |

To a solution of Pro-Hydrocodone in DMF was added NMM followed by Boc-Ala-OSu. The solution was stirred at ambient temperatures for 18 hours. Solvent was removed. Crude material was purified using preparative HPLC (Phenomenex Luna C18, 30×250 mm, 5 μM, 100 Å; Gradient: 100 water/O 0.1% TFA-MeCN→0/100; 30 ml/min.). Solid was collected as a slightly yellow powder (0.307 g, 85% yield): ¹H NMR (DMSO-d₆) δ 1.16 (d, 3H), 1.35 (s, 9H), 1.51 (m, 2H), 1.86-2.10 (m, 6H), 2.50 (m, 1H), 2.54 (m, 1H), 2.69 (m, 1H), 2.88 (s, 3H), 3.02 (dd, 1H), 3.26 (d, 1H), 3.55 (m, 1H), 3.67 (m, 1H), 3.72 (s, 3H), 3.80 (s, 1H), 4.25 (m, 1H), 4.43 (d, 1H), 5.01 (s, 1H), 5.59 (d, 1H), 6.75 (d, 1H), 6.88 (d, 1H), 6.99 (t, 1H), 9.91 (br s, 1H).

To the Boc-Ala-Pro-Hydrocodone (0.100 g) was added 10 ml of 4N HCl in dioxane. The resulting mixture was stirred at ambient temperatures for 18 hours. Solvent was removed and final product dried under vacuum. Solid was collected as a slightly yellow solid (0.56 g, 71% yield): ¹H NMR (DMSO-d₆) δ 1.38 (s, 3H), 1.48 (t, 1H), 1.80-2.29 (m, 8H), 2.65 (m, 1H), 2.80 (s, 3H), 2.96 (m, 3H), 3.23 (m, 2H), 3.76 (s, 3H), 3.92 (s, 1H), 4.22 (s, 1H), 4.53 (s, 1H), 5.00 (s, 1H), 5.84 (d, 1H), 6.77 (d, 1H), 6.86 (d, 1H), 8.25 (br s, 3H).

### Example 3

### Example of Conjugates Containing Two Identical Amino acids Glu-Glu-Hydrocodone

Glu-Glu-Hydrocodone was prepared by a similar method to Example 2 except the amino acid starting material was Boc-Glu(OtBu)-OSu and the conjugate starting material was Glu-Hydrocodone.

Tripeptide Hydrocodone Conjugates

### Example 4

### Example of Conjugates Containing Different Amino Acids: Gly-Gly-Leu-Hydrocodone

| Reagents | MW | Weight | | mmoles | Molar Equivalents |
|---|---|---|---|---|---|
| Leu-Hydrocodone | 484 | 2.21 | g | 4.56 | 1.0 |
| Boc-Gly-Gly-OSu | 329 | 3.00 | g | 9.12 | 2.0 |
| NMM | 101 | 5.0 | ml | 45.6 | 10 |
| DMF | — | 100 | ml | — | — |

To a solution of Leu-Hydrocodone in DMF was added NMM followed by Boc-Gly-Gly-OSu. The solution was stirred at ambient temperatures for 18 hours. Solvent was removed. Crude material was purified using preparative HPLC (Phenomenex Luna C18, 30×250 mm, 5M, 100 Å; Gradient: 90 water/10 0.1% TFA-MeCN→0/100; ml/min.). Solid was collected as a slightly yellow powder (2.08 g, 73% yield): ¹H NMR (DMSO-d₆) δ 0.88 (dd, 6H), 1.38 (s, 9H), 1.53-1.72 (m, 5H), 1.89 (d, 1H), 2.15 (m, 1H), 2.67 (m, 2H), 2.94 (s, 3H), 3.05 (m, 2H), 3.25 (m, 2H), 3.56 (d, 3H), 3.76 (s, 6H), 3.98 (s, 1H), 4.35 (q, 1H), 5.04 (s, 1H), 5.59 (d, 1H), 6.77 (d, 1H), 6.85 (d, 1H), 7.04 (t, 1H), 8.01 (t, 1H), 8.30 (d, 1H), 9.99 (br s, 1H).

To the Boc-Gly-Gly-Leu-Hydrocodone (2.08 g) was added 50 ml of 4N HCl in dioxane. The resulting mixture was stirred at ambient temperatures for 18 hours. Solvent was removed and final product dried under vacuum. Solid was collected as a slightly yellow solid (1.72 g, 86% yield): ¹H NMR (DMSO-d₆) δ 0.89 (dd, 6H), 1.50-1.87 (m, 5H), 2.26 (m, 2H), 2.66 (m, 2H), 2.82-2.97 (m, 5H), 3.21 (m, 2H), 3.60 (m, 4H), 3.88 (m, 5H), 4.37 (m, 1H), 5.04 (s, 1H), 5.60 (s, 1H), 6.79 (d, 2H), 8.07 (br s, 3H), 8.54 (br s, 1H), 8.66 (br s, 1H), 11.29 (br s, 1H).

### Example 5

### Example of Conjugates Containing Three Identical Amino Acids: Glu-Glu-Glu-Hydrocodone

Glu-Glu-Glu-Hydrocodone was prepared by a similar method to Example 4 except the amino acid starting material was Boc-Glu(OtBu)-Glu(OtBu)-OSu and the conjugate starting material was Glu-Hydrocodone.

Pentapeptide Hydrocodone Conjugates

### Example 8

### Example of Conjugates Containing Different Amino Acids: Gly-Gly-Gly-Gly-Leu-Hydrocodone

| Reagents | MW | Weight | | mmoles | Molar Equivalents |
|---|---|---|---|---|---|
| Gly-Gly-Leu- | 599 | 0.580 | g | 0.970 | 1.0 |
| Hydrocodone | | | | | |
| Boc-Gly-Gly-OSu | 329 | 0.638 | g | 1.94 | 2.0 |
| NMM | 101 | 1.06 | ml | 9.70 | 10 |
| DMF | — | 20 | ml | — | — |

To a solution of Gly-Gly-Leu-Hydrocodone in DMF was added NMM followed by Boc-Gly-Gly-OSu. The solution was stirred at ambient temperatures for 18 hours. Solvent was removed. Crude material was purified using preparative HPLC (Phenomenex Luna C18, 30×250 mm, 5M, 100 Å; Gradient: 85 water/15 0.1% TFA-MeCN→50/50; 30 ml/min.). Solid was collected as a slightly yellow powder (0.304 g, 37% yield).

To the Boc-Gly-Gly-Gly-Gly-Leu-Hydrocodone (0.304 g) was added 25 ml of 4N HCl in dioxane. The resulting mixture was stirred at ambient temperatures for 18 hours. Solvent was removed and final product dried under vacuum. Solid was collected as a slightly yellow solid (0.247 g, 97% yield): ¹H NMR (DMSO-d₆) δ 0.87 (m, 6H), 1.23 (s, 1H), 1.51-1.86 (m, 4H), 2.18 (m, 1H), 2.71 (m, 2H), 2.77 (s, 3H), 2.96 (m, 2H), 3.17 (m, 2H), 3.61 (s, 3H), 3.81-3.84 (m, 10H), 4.22 (m, 1H), 4.36 (m, 1H), 5.09 (m, 1H), 5.59 (d, 1H), 6.74 (dd, 2H), 8.16 (br s, 4H), 8.38 (br s, 1H), 8.74 (br s, 1H), 11.42 (br s, 1H).

### Example 9

### Example of Conjugates Containing Different Amino Acids Glu-Glu-Gly-Gly-Ile-Hydrocodone

Glu-Glu-Gly-Gly-Ile-Hydrocodone was prepared by a similar method to Example 8 except the amino acid starting material was Boc-Glu(OtBu)-Glu(OtBu)-OSu and the conjugate starting material was Gly-Gly-Ile-Hydrocodone.

### Example 10

### Example of Conjugates Containing Different Amino Acids Gly-Gly-Gly-Gly-Ile-Hydrocodone

Gly-Gly-Gly-Gly-Ile-Hydrocodone was prepared by a similar method to Example 8 except the amino acid starting material was Boc-Gly-Gly-OSu and the conjugate starting material was Gly-Gly-Ile-Hydrocodone.

### Example 11

### Example of Conjugates Containing Different Amino Acids Glu-Glu-Phe-Phe-Phe-Hydrocodone

Glu-Glu-Phe-Phe-Phe-Hydrocodone was prepared by a similar method to Example 8 except the amino acid starting material was Boc-Glu(OtBu)-Glu(OtBu)-OSu and the conjugate starting material was Phe-Phe-Phe-Hydrocodone.

### Example 12

### Example of Conjugates Containing Different Amino Acids Tyr-Tyr-Phe-Pro-Ile-Hydrocodone

Tyr-Tyr-Phe-Pro-Ile-Hydrocodone was prepared by a similar method to Example 8 except the amino acid starting material was Boc-Tyr(tBu)-Tyr(tBu)-OSu and the conjugate starting material was Phe-Pro-Ile-Hydrocodone.

### Example 13

### Example of Conjugates Containing Five Identical Amino Acids: Glu-Glu-Glu-Glu-Glu-Hydrocodone

Glu-Glu-Glu-Glu-Glu-Hydrocodone was prepared by a similar method to Example 8 except the amino acid starting material was Boc-Glu(OtBu)-Glu(OtBu)-OSu and the conjugate starting material was Glu-Glu-Glu-Hydrocodone.

Glycopeptide Hydrocodone Conjugates

| Reagents | MW | Weight | | mmoles | Molar Equivalents |
|---|---|---|---|---|---|
| 1,2:3,4-di-O- | 260 | 1.00 | g | 3.85 | 1 |
| isopropylidene-D- | | | | | |
| galactopyranose | | | | | |
| 20% Phosgene | — | 20 | ml | — | — |
| in toluene | | | | | |

### Example 14

### Chloroformate of 1,2:3,4-di-O-isopropylidene-D-galactopyranose

To a stirring solution of 20% phosgene in toluene under an inert atmosphere was added 1,2:3,4-di-O-isopropylidene-D-galactopyranose via syringe. The resulting clear, colorless solution was stirred at ambient temperature for 30 minutes. After stirring, Ar(g) was bubbled through the solution for approximately 20 minutes to remove any excess phosgene. Solvent was then removed and product dried under vacuum for 18 hours. Product was used without further purification or characterization.

### Example 15

### Galactose-CO-Leu-Hydrocodone

To the chloroformate of galactose (1.5 eq) in dimethylformamide (DMF) (2 ml/mmol) was added Leu-Hydrocodone (1 eq) and 4-methylmorpholine (NMM) (6 eq). The reaction was stirred at ambient temperatures for 18 hours. Reaction was quenched by the addition of water, solvents were removed and crude product was isolated by purification with reverse-phase HPLC.

Product was deprotected using 1:1 μM HCl: THF (1 ml/0.1 mmol) in 3 hours. Product was re-purified by reverse-phase HPLC.

### Example 16

### Galactose-CO-Pro-Pro-Ile-Hydrocodone

Galactose-CO-Pro-Pro-Ile-Hydrocodone was prepared in a manner similar to Example 15 except Pro-Pro-Ile-Hydrocodone was used as the conjugated starting material.

### Example 17

### Gulonic acid-Ile-Hydrocodone

Gulonic acid-Ile-Hydrocodone was prepared in a manner similar to Example 15 except Ile-Hydrocodone was used as the conjugated starting material and Gulonic acid-OSu was used as the carbohydrate starting material.

D-amino Acid Hydrocodone Conjugates

### Example 18

### (d)-Lys-(1)-Lys-Ile-Hydrocodone

To a solution of Ile-Hydrocodone in DMF was added NMM followed by Boc-(d)-Lys(Boc)-(1)-Lys(Boc)-OSu. The solution was stirred at ambient temperatures for 18 hours. Solvent was removed. Crude material was purified using preparative HPLC (Phenomenex Luna C18, 30×250 mm, 5 μM, 100 Å; Gradient: 90 water/10 0.1% TFA-MeCN→0/100; 30 ml/min.). Solid was collected as a slightly yellow powder. To the Boc-(d)-Lys(Boc)-(1)-Lys(Boc)-Hydrocodone was added 4N HCl in dioxane. The resulting mixture was stirred at ambient temperatures for 18 hours. Solvent was removed and final product dried under vacuum. Solid was collected as a slightly yellow solid.

Oral Bioavailability of Peptide-Hydrocodone Conjugates at a Dose (1 mg/kg) Approximating a Therapeutic Human Dose and at an Elevated Dose

When the peptides are conjugated to the active agent hydrocodone oral bioavailability is maintained or increased over an equivalent hydrocodone dose when the dose is administered as 1 mg/kg. This dose is the equivalent of a human dose of 10 to 14 mg for an individual weighing 70 kg (148 lbs) according to Chou et al. However, when administered orally at 5 mg/kg peak levels and bioavailability of are substantially decreased. A 5 mg/kg dose in rats approximates an 80 mg human equivalent dose (HED) of hydrocodone bitartrate; a dose that would be likely to be harmful to a naïve patient in immediate release form with the potential for fatal overdose. Human equivalent doses are defined as the equivalent dose for a 60 kg person adjusted for the body surface area of the animal model. The adjustment factor for rats is 6.2. The HED for a rat dose of 5 mg/kg of hydrocodone base, for example, is equivalent to 48.39 mg (5/6.2×60) hydrocodone base; which is equivalent to 79.98 (48.39/0.605) mg hydrocodone bitartrate, when adjusted for the salt content.

Thus the peptide-hydrocodone conjugates maintain their therapeutic value at the lower dose (1 mg/kg), whereas when given at a dose above a safe level (5 mg/kg) bioavailability is decreased as compared to hydrocodone, thus diminishing the potential for overdose by oral ingestion. The decrease in bioavailability of hydrocodone from peptide hydrocodone conjugates relative to hydrocodone ranged from 9 to 70 percent.

### Example 20

### Bioavailability of Peptide-HC Conjugates by the Intranasal Route

When the peptides are conjugated to the active agent hydrocodone the bioavailability by the intravenous route is substantially decreased thereby diminishing the possibility of overdose when the drug is administered by snorting.

### Example 21

### Hydrocodone Conjugates

Bioavailability (AUC and Cmax) of various peptide-hydrocodone conjugates relative to that of hydrocodone bitartrate have been studied. At the relatively low doses of 1 and 2 mg/kg (human equivalent doses (HEDs) of 16 and 32 mg hydrocodone bitartrate) hydrocodone conjugates show comparable bioavailability to that of hydrocodone bitartrate. At the elevated doses of 5 and 25 mg/kg bioavailability of hydrocodone and hydromorphone were substantially decreased as compared to that of hydrocodone. These doses (HED of 80 and 400 mg hydrocodone bitartrate) are equivalent to amounts well above the available prescription doses of hydrocodone bitartrate which range from 2.5 to 10 mg. When delivered by the parenteral routes of intravenous and intranasal administration a substantial decrease in bioavailability of hydrocodone and hydromorphone from hydrocodone conjugates as compared to hydrocodone bitartrate was observed. These examples establish that covalent modification of an opioid (HC) via attachment of a peptide provides a method of delivering bioequivalent doses when given at doses approximating a normal prescribed dose. When administered by parenteral routes or at oral doses in excess of the intended prescription the bioavailability is substantially decreased. Collectively, the examples clearly illustrate the utility of the invention for decreasing the abuse potential of opioids.

Summary of in vivo testing of abuse resistant hydrocodone conjugates. In vivo testing of hydrocodone conjugates demonstrates for instance decreased intranasal analgesic response, decreased intravenous analgesic response, decreased subcutaneous analgesic response, decreased oral Cₘₐₓ, decreased intranasal bioavailability (AUC and Cₘₐₓ), and decreased intravenous bioavailability (AUC and Cₘₐₓ) of hydrocodone conjugates and is described in further detail below.

### Example 22

### Decreased Intranasal Analgesic Response to Hydrocodone Conjugates

Male Sprague-Dawley rats were dosed by placing 0.02 ml of water containing hydrocodone conjugate or hydrocodone bitartrate into the nasal flares. All doses contained equivalent amounts of hydrocodone base. The time (seconds) until paw lick latency was used a measure of the analgesic effect. Rats were habituated to determine baseline response. Hot plate tests were conducted at 55° C. A limit of 45 seconds was used in all testing to avoid tissue damage. All animals were humanely sacrificed following the end of testing. The paw lick latency (analgesic effect)-time curves shown in FIGS. 61 and 63 indicate the decrease in analgesia produced by the hydrocodone conjugates as compared to an equimolar (hydrocodone base) dose of hydrocodone bitartrate. The analgesic response as determined by the hot plate test is a pharmacodynamic measurement of the pharmacological effect of hydrocodone. These examples illustrate that hydrocodone conjugates decrease the analgesic effect by the intranasal route of administration as compared to hydrocodone bitartrate.

### Example 23

### Decreased Intravenous Analgesic Response to Hydrocodone Conjugates

Male Sprague-Dawley rats were dosed by tail vein injection of 0.1 ml of water containing hydrocodone conjugates or hydrocodone bitartrate. All doses contained equivalent amounts of hydrocodone base. The time (seconds) until paw lick latency was used a measure of the analgesic effect. Rats were habituated to determine baseline response. Hot plate tests were conducted at 55° C. A limit of 45 seconds was used in all testing to avoid tissue damage. All animals were humanely sacrificed following the end of testing. The paw lick latency (analgesic effect)-time curve shown in FIG. 16 indicates the decrease in analgesia produced by a hydrocodone conjugate as compared to an equimolar (hydrocodone base) dose of hydrocodone bitartrate. The analgesic response as determined by the hot plate test is a pharmacodynamic measurement of the pharmacological effect of hydrocodone. This example illustrates that a hydrocodone conjugate decreased the analgesic effect by the intravenous route of administration as compared to hydrocodone bitartrate.

### Example 24

### Decreased Subcutaneous Analgesic Response to Hydrocodone Conjugates

Male Sprague-Dawley rats were dosed by subcutaneous injection of 0.1 ml of water containing hydrocodone conjugates or hydrocodone bitartrate. All doses contained equivalent amounts of hydrocodone base. The time (seconds) until paw lick latency was used a measure of the analgesic effect. Rats were habituated to determine baseline response. Hot plate tests were conducted at 55° C. A limit of 45 seconds was used in all testing to avoid tissue damage. All animals were humanely sacrificed following the end of testing. The paw lick latency (analgesic effect)-time curve indicates the decrease in analgesia produced by a hydrocodone conjugate as compared to an equimolar (hydrocodone base) dose of hydrocodone bitartrate. The analgesic response as determined by the hot plate test is a pharmacodynamic measurement of the pharmacological effect of hydrocodone. This example illustrates that a hydrocodone conjugate decreased the analgesic effect by the subcutaneous route of administration as compared to hydrocodone bitartrate.

### Example 25

### Decreased Oral Cₘₐₓ of Hydrocodone Conjugates

Male Sprague-Dawley rats were provided water ad libitum, fasted overnight and dosed by oral gavage with hydrocodone conjugates or hydrocodone bitartrate. All doses contained equivalent amounts of hydrocodone base. Plasma hydrocodone concentrations were measured by ELISA (Hydromorphone, 106619-1, Neogen, Corporation, Lexington, Ky.) and/or LC/MS. The assay is specific for hydromorphone (the major hydrocodone metabolite, 100% reactive) and hydrocodone (62.5% reactive). These examples illustrate that hydrocodone conjugates decrease the peak level (Cₘₐₓ) of hydrocodone plus hydromorphone as compared to that produced by equimolar (hydrocodone base) doses of hydrocodone bitartrate when given by the oral route of administration.

### Example 26

### Decreased Intranasal Bioavailability (AUC and Cₘₐₓ) Hydrocodone Conjugates

Male Sprague-Dawley rats were provided water ad libitum and doses were administered by placing 0.02 ml of water containing hydrocodone conjugates or hydrocodone bitartrate into the nasal flares. All doses contained equivalent amounts of hydrocodone base. Plasma hydrocodone concentrations were measured by ELISA (Hydromorphone, 106619-1, Neogen, Corporation, Lexington, Ky.) and/or LC/MS. The assay is specific for hydromorphone (the major hydrocodone metabolite, 100% reactive) and hydrocodone (62.5% reactive). These examples illustrate that hydrocodone conjugates decrease the peak level (Cₘₐₓ) and total absorption (AUC) of hydrocodone plus hydromorphone as compared to those produced by equimolar (hydrocodone base) doses of hydrocodone bitartrate when given by the intranasal route of administration.

### Example 27

### Decreased Intravenous Bioavailability (AUC and Cₘₐₓ) Hydrocodone Conjugates

Male Sprague-Dawley rats were provided water ad libitum and doses were administered by intravenous tail vein injection of 0.1 ml of water containing hydrocodone conjugates or hydrocodone bitartrate. All doses contained equivalent amounts of d-amphetamine base. Plasma hydrocodone concentrations were measured by ELISA (Hydromorphone, 106619-1, Neogen, Corporation, Lexington, Ky.) and/or LC/MS. The assay is specific for hydromorphone (the major hydrocodone metabolite, 100% reactive) and hydrocodone (62.5% reactive). This example illustrates that a dose of hydrocodone conjugate decreases the peak level (Cₘₐₓ) and total absorption (AUC) of hydrocodone plus hydromorphone as compared to those produced by an equimolar (hydrocodone base) dose of hydrocodone bitartrate when given by the intranasal route of administration.

TABLE 3
Oral and Intranasal Bioavailability of Hydrocodone conjugates.

| | | |
|---|---|---|
| stability | bioavailability | |
| 90° C., | (% HC) | Oral:IN |

| | | | |
|---|---|---|---|
| 20 min | oral | IN | Index |

| | | | | | | | | | | | |
|---|---|---|---|---|---|---|---|---|---|---|---|
| Compound Class | Compound | V | BP | TW | BS | AUC | Cmax | AUC | Cmax | AUC | Cmax |

Oral AUC >80%

| | | | | | | | | | | | |
|---|---|---|---|---|---|---|---|---|---|---|---|
| Tripeptide | Gly-Gly-Leu-HC | 1 | 100 | 85 | 100 | 82 | 126 | 77 | 62 | 1.06 | 2.03 |
| Tripeptide | Gly-Gly-Ile-HC | 0 | 100 | 93 | 100 | 95 | 167 | 93 | 103 | 1.02 | 1.62 |
| Tripeptide | Leu-Pro-Phe-HC | 2 | 100 | 100 | 100 | 106 | 125 | 83 | 101 | 1.28 | 1.24 |
| Tripeptide | Pro-Pro-Ile-HC | 0 | 16 | 70 | 100 | 112 | 99 | 59 | 65 | 1.90 | 1.52 |
| Tripeptide | Pro-Pro-Leu-HC | 2 | 100 | 100 | 100 | 94 | 108 | 46 | 48 | 2.04 | 2.25 |
| Tripeptide | Pro-Ile-Ile-HC | 0 | 47 | 83 | 100 | 104 | 99 | 86 | 102 | 1.21 | 0.97 |
| Tripeptide | Glu-Glu-Ile-HC | 0 | 94 | 26 | 100 | 83 | 112 | 52 | 59 | 1.60 | 1.90 |
| Tripeptide | Tyr-Tyr-Ile-HC | 0 | 66 | 23 | 100 | 145 | 234 | 20 | 34 | 7.25 | 6.88 |
| Tripeptide | Lys-Lys-Ile-HC | 0 | 100 | 96 | 97 | 80 | 76 | 68 | 94 | 1.18 | 0.81 |
| Tripeptide | Asp-Asp-Ile-HC | 0 | 40 | 10 | 100 | 280 | 238 | 59 | 93 | 4.75 | 2.56 |
| Tripeptide | Pro-Leu-Ile-HC | 0 | 100 | 100 | 100 | 141 | 172 | 87 | 101 | 1.62 | 1.70 |
| Tripeptide | (d)Lys(I)Lys-Ile-HC | 0 | 69 | 74 | 100 | 141 | 174 | 41 | 54 | 3.44 | 3.22 |
| Pentapeptide | Glu-Glu-Gly-Gly-Phe-HC | 0 | 100 | 100 | 100 | 110 | 112 | 89 | 97 | 1.24 | 1.15 |
| Pentapeptide | Glu-Glu-Gly-Gly-Ile-HC | 0 | 99 | 23 | 99 | 81 | 77 | 50 | 56 | 1.62 | 1.38 |
| Pentapeptide | Glu-Glu-Phe-Phe-Ile-HC | 0 | 100 | 33 | 100 | 96 | 129 | 68 | 76 | 1.41 | 1.70 |
| Pentapeptide | Glu-Glu-Phe-Phe-Phe-HC | 3 | 100 | 57 | 84 | 83 | 89 | 27 | 47 | 3.07 | 1.89 |
| Pentapeptide | Lys-Lys-Pro-Pro-Ile-HC | 0 | 72 | 66 | 100 | 80 | 76 | 68 | 94 | 1.18 | 0.81 |
| Pentapeptide | Tyr-Tyr-Pro-Pro-Ile-HC | 0 | 100 | 83 | 100 | 218 | 213 | 10 | 10 | NA | NA |
| Pentapeptide | Asp-Asp-Pro-Pro-Ile-HC | 0 | 75 | 11 | 100 | 92 | 95 | 45 | 80 | 2.04 | 1.19 |
| Pentapeptide | Asp-Asp-Gly-Gly-Ile-HC | 0 | 68 | 3 | 100 | 82 | 80 | 48 | 67 | 1.71 | 1.19 |
| Pentapeptide | Gly-Gly-Pro-Pro-Ile-HC | 0 | 73 | 70 | 100 | 94 | 121 | 44 | 56 | 2.14 | 2.16 |
| Pentapeptide | Tyr-Tyr-Phe-Phe-Ile-HC | 0 | 5 | 5 | 50 | 113 | 63 | 26 | 34 | 4.35 | 1.85 |
| Pentapeptide | Asp-Asp-Phe-Phe-Ile-HC | 0 | 73 | 14 | 94 | 115 | 167 | 56 | 62 | 2.05 | 2.69 |
| Pentapeptide | Glu-Glu-Asp-Asp-Ile-HC | 1 | 77 | 15 | 100 | 108 | 129 | 53 | 81 | 2.04 | 1.59 |
| Pentapeptide | Lys-Lys-Asp-Asp-Ile-HC | 0 | 64 | 0 | 100 | 90 | 121 | 39 | 56 | 2.31 | 2.16 |
| Pentapeptide | Asp-Asp-Asp-Asp-Ile-HC | 2 | 32 | 2 | 99 | 79 | 110 | 36 | 64 | 2.19 | 1.72 |
| Pentapeptide | Gly-Gly-Glu-Glu-Ile-HC | 0 | 74 | 11 | 100 | 96 | 119 | 66 | 77 | 1.45 | 1.55 |

Oral AUC >80%; IN AUC <60%

| | | | | | | | | | | | |
|---|---|---|---|---|---|---|---|---|---|---|---|
| Tripeptide | Pro-Pro-Ile-HC | 0 | 16 | 70 | 100 | 112 | 99 | 59 | 65 | 1.90 | 1.52 |
| Tripeptide | Pro-Pro-Leu-HC | 2 | 100 | 100 | 100 | 94 | 108 | 46 | 48 | 2.04 | 2.25 |
| Tripeptide | Glu-Glu-Ile-HC | 0 | 94 | 26 | 100 | 83 | 112 | 52 | 59 | 1.60 | 1.90 |
| Tripeptide | Tyr-Tyr-Ile-HC | 0 | 66 | 23 | 100 | 145 | 234 | 20 | 34 | 7.25 | 6.88 |
| Tripeptide | Asp-Asp-Ile-HC | 0 | 40 | 10 | 100 | 280 | 238 | 59 | 93 | 4.75 | 2.56 |
| Tripeptide | (d)Lys(I)Lys-Ile-HC | 0 | 69 | 74 | 100 | 141 | 174 | 41 | 54 | 3.44 | 3.22 |
| Pentapeptide | Glu-Glu-Gly-Gly-Ile-HC | 0 | 99 | 23 | 99 | 81 | 77 | 50 | 56 | 1.62 | 1.38 |
| Pentapeptide | Glu-Glu-Phe-Phe-Phe-HC | 3 | 100 | 57 | 84 | 83 | 89 | 27 | 47 | 3.07 | 1.89 |
| Pentapeptide | Tyr-Tyr-Pro-Pro-Ile-HC | 0 | 100 | 83 | 100 | 218 | 213 | IC | IC | NA | NA |
| Pentapeptide | Asp-Asp-Pro-Pro-Ile-HC | 0 | 75 | 11 | 100 | 92 | 95 | 45 | 80 | 2.04 | 1.19 |
| Pentapeptide | Asp-Asp-Gly-Gly-Ile-HC | 0 | 68 | 3 | 100 | 82 | 80 | 48 | 67 | 1.71 | 1.19 |
| Pentapeptide | Gly-Gly-Pro-Pro-Ile-HC | 0 | 73 | 70 | 100 | 94 | 121 | 44 | 56 | 2.14 | 2.16 |
| Pentapeptide | Tyr-Tyr-Phe-Phe-Ile-HC | 0 | 5 | 5 | 50 | 113 | 63 | 26 | 34 | 4.35 | 1.85 |
| Pentapeptide | Asp-Asp-Phe-Phe-Ile-HC | 0 | 73 | 14 | 94 | 115 | 167 | 56 | 62 | 2.05 | 2.69 |
| Pentapeptide | Glu-Glu-Asp-Asp-Ile-HC | 1 | 77 | 15 | 100 | 108 | 129 | 53 | 81 | 2.04 | 1.59 |
| Pentapeptide | Lys-Lys-Asp-Asp-Ile-HC | 0 | 64 | 0 | 100 | 90 | 121 | 39 | 56 | 2.31 | 2.16 |
| Pentapeptide | Asp-Asp-Asp-Asp-Ile-HC | 2 | 32 | 2 | 99 | 79 | 110 | 36 | 64 | 2.19 | 1.72 |
| Tripeptide | Glu-Glu-Ile-HC | 0 | 94 | 26 | 100 | 83 | 112 | 52 | 59 | 1.60 | 1.90 |
| Tripeptide | Tyr-Tyr-Ile-HC | 0 | 66 | 23 | 100 | 145 | 234 | 20 | 34 | 7.25 | 6.88 |
| Tripeptide | Asp-Asp-Ile-HC | 0 | 40 | 10 | 100 | 280 | 238 | 59 | 93 | 4.75 | 2.56 |
| Pentapeptide | Glu-Glu-Gly-Gly-Ile-HC | 0 | 99 | 23 | 99 | 81 | 77 | 50 | 56 | 1.62 | 1.38 |
| Pentapeptide | Asp-Asp-Pro-Pro-Ile-HC | 0 | 75 | 11 | 100 | 92 | 95 | 45 | 80 | 2.04 | 1.19 |
| Pentapeptide | Asp-Asp-Gly-Gly-Ile-HC | 0 | 68 | 3 | 100 | 82 | 80 | 48 | 67 | 1.71 | 1.19 |
| Pentapeptide | Tyr-Tyr-Phe-Phe-Ile-HC | 0 | 5 | 5 | 50 | 113 | 63 | 26 | 34 | 4.35 | 1.85 |
| Pentapeptide | Asp-Asp-Phe-Phe-Ile-HC | 0 | 73 | 14 | 94 | 115 | 167 | 56 | 62 | 2.05 | 2.69 |
| Pentapeptide | Glu-Glu-Asp-Asp-Ile-HC | 1 | 77 | 15 | 100 | 108 | 129 | 53 | 81 | 2.04 | 1.59 |
| Pentapeptide | Lys-Lys-Asp-Asp-Ile-HC | 0 | 64 | 0 | 100 | 90 | 121 | 39 | 56 | 2.31 | 2.16 |
| Pentapeptide | Asp-Asp-Asp-Asp-Ile-HC | 2 | 32 | 2 | 99 | 79 | 110 | 36 | 64 | 2.19 | 1.72 |

Collectively, the examples illustrate the application of the invention for reducing the overdose potential of hydrocodone. These examples establish that hydrocodone can be covalently modified by attachment of a chemical moiety in a manner that maintains therapeutic value over a normal dosing range, while substantially decreasing if not eliminating the possibility of overdose by oral, intranasal, or intravenous routes of administration with the hydrocodone.

1. A compound of the formula: 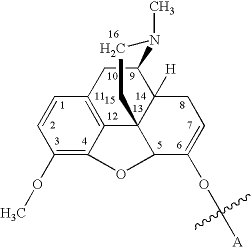
wherein A is a carrier peptide, and a pharmaceutically acceptable salt thereof.

2. The compound of claim 1, wherein said carrier peptide is selected from an amino acid, a dipeptide, a tripeptide, a tetrapeptide and a pentapeptide.

3. The compound of claim 1, wherein said carrier peptide is selected from acetyl-Glu-Glu-Pro-Pro-Ile, Asp-Asp-Gly-Gly-Ile, Asp-Asp-Leu-Leu-Ile, Asp-Asp-Leu-Leu-Ile, Asp-Asp-Pro-Pro-Ile, Ethyl Carbonate, galactose-Gly-Gly-Ile, galactose-Gly-Gly-Leu, galactose-Ile, Glu-Glu-Gly-Gly-Phe, Glu-Glu-Leu-Leu-Leu, Glu-Glu-Phe-Phe-Leu, Glu-Glu-Phe-Pro-Ile, Glu-Glu-Pro-Pro-Leu, Glu-Glu-Pro-Phe-Ile, Glu-Glu-Glu-Glu-Ile, Glu_{pyro}-Glu, Gly-Gly-Glu-Glu-Ile, Lys-Lys-Leu-Leu-Ile, Lys-Lys-Pro-Pro-Ile, Phe-Phe-Glu-Glu-Ile, Phe-Phe-Phe-Phe-Phe, Thr-Thr-Gly-Gly-Ile, Thr-Thr-Phe-Phe-Ile, Tyr-Tyr-Leu-Leu-Ile, Tyr-Tyr-Phe-Phe-Ile, Tyr-Tyr-Pro-Pro-Ile, Tyr-Tyr-Pro-Phe-Ile, Tyr-Tyr-Phe-Phe-Ile, and Glu-Glu-Phe-Phe-Phe.

4. The compound of claim 2, wherein said carrier peptide is a tripeptide selected from (D)Lys-Lys-Ile, Asp-Asp-Ile, Gln-Gln-Ile, Glu-Glu-Leu, Gly-Ile-Ile, Leu-Leu-Ile, Leu-Pro-Ile, Lys-Lys-Ile, Phe-Phe-Ile, Phe-Phe-Leu, Phe-Phe-Phe, Pro-Ile-Ile, Pro-Leu-Ile, Pro-Phe-Ile, Thr-Thr-Ile, Tyr-Tyr-Ile, Gln-Gln-Ile, Tyr-Tyr-Ile, Asp-Asp-Ile, and Pro-Pro-Leu.

5. A pharmaceutical composition comprising a compound of the formula: 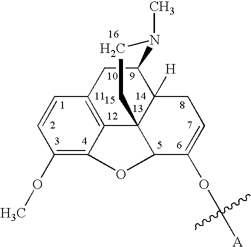
wherein A is a carrier peptide or a pharmaceutically acceptable salt thereof, and a pharmaceutically acceptable excipient.

6. The composition of claim 5, wherein said carrier peptide is selected from an amino acid, a dipeptide, a tripeptide, a tetrapeptide and a pentapeptide.

7. The composition of claim 5 which provides a serum release curve for hydrocodone that does not increase above the toxicity level of hydrocodone when taken at doses exceeding those within the therapeutic range for unbound hydrocodone.

8. The composition of claim 5 which maintains a steady-state serum release curve of hydrocodone that provides a therapeutically effective bioavailability but prevents spiking or increased blood serum concentrations compared to unbound hydrocodone.

9. The composition of claim 5, wherein when said composition is administered orally, bioavailability of hydrocodone or a salt thereof is maintained, but when administered intravenously or intranasally, the bioavailability of hydrocodone is decreased.

10. The composition of claim 5 which is in a form suitable for oral administration.

11. A method of treating pain, comprising administering to a patient in need thereof a therapeutically effective amount of a compound of the formula: 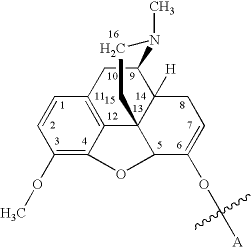
wherein A is a carrier peptide, and a pharmaceutically acceptable salt thereof.

12. The method of claim 11, wherein said carrier peptide is selected from an amino acid, a dipeptide, a tripeptide, a tetrapeptide and a pentapeptide.
